(19) United States
(12) Patent Application Publication
(10) Pub. No.: US 2008/0200515 A1
(43) Pub. Date: Aug. 21, 2008

(54) SOLID STATE FORMS OF ENANTIOPURE ILAPRAZOLE

(75) Inventors: John M. BRACKETT, Kenosha, WI (US); David T. JONAITIS, Lafayette, IN (US); Wei LAI, West Lafayette, IN (US); Jih Hua LIU, Green Oaks, IL (US); Stephan D. PARENT, West Lafayette, IN (US)

Correspondence Address: NIXON PEABODY, LLP, 401 9TH STREET, NW, SUITE 900, WASHINGTON, DC, 20004-2128, US

(73) Assignee: TAP PHARMACEUTICAL PRODUCTS INC., Lake Forest, IL (US)

(21) Appl. No.: 11/966,808

(22) Filed: Dec. 28, 2007

Related U.S. Application Data
(60) Provisional application No. 60/877,607, filed on Dec. 29, 2006.

(51) Int. Cl. A61K 31/4439; C07D 401/14; A61P 1/00

(52) U.S. Cl. 514/338; 546/273.7

(57) ABSTRACT

The invention relates to solid state forms of enantiopure ilaprazole, 2[[(4-methoxy-3-methyl-2-pyridinyl)-methyl]sulfnyl]-5-(1H-pyrrol-1-yl) 1H-Benzimidazole. The invention also relates to a pharmaceutical composition for inhibiting gastric acid secretion comprising a solid form of ilaprazole according to the invention in an amount effective to inhibit gastric acid secretion and a pharmaceutically acceptable carrier. The invention also provides methods of treatment for various acid-related gastrointestinal (GI) disorders such as those discussed above.

### PRIORITY STATEMENT

This application claims benefit under 35 U.S.C. § 119(e) of U.S. Provisional Application No. 60/877,607, filed Dec. 29, 2006, which is herein incorporated by reference in its entirety.

### FIELD OF THE INVENTION

This invention relates to ilaprazole, 2[[(4-methoxy-3-methyl-2-pyridinyl)-methyl]sulfinyl]-5-(1H-pyrrol-1-yl) 1H-Benzimidazole, a substituted benzimidazole having a chiral sulfur atom. More particularly, the invention relates to solid state forms of enantiopure ilaprazole. Ilaprazole is a proton pump inhibitor and is useful in the treatment of various acid-related gastrointestinal disorders.

### BACKGROUND OF THE INVENTION

Since their introduction in the late 1980s, proton pump inhibitors have improved the treatment of various acid-related gastrointestinal (GI) disorders, including gastroesophageal reflux disease (GERD), peptic ulcer disease, Zollinger-Ellison Syndrome (ZES), ulcers, and nonsteroidal anti-inflammatory drug (NSAID)-induced gastropathy. GERD encompasses three disease categories: non-erosive reflux disease (NERD), erosive esophagitis, and Barret' esophagus. ZES is caused by a gastrin-secreting tumor of the pancreas that stimulates the acid-secreting cells of the stomach to maximal activity. Proton pump inhibitors have also been used to treat ulcers such as duodenal, gastric, and NSAID-associated gastric/duodenal ulcers.

As antisecretory drugs, proton pump inhibitors are currently the recommended first line therapy, being viewed as more effective than other treatments. In general, proton pump inhibitors offer superior gastric acid suppression over histamine H2-receptor blockers. The use of proton pump inhibitors by patients who suffer from gastric acid-related disorders is generally believed to have led to an increase in their quality of life, productivity, and overall well being.

Proton pump inhibitors are also used to treat extra-esophageal manifestations of GERD (asthma, hoarseness, chronic cough, non-cardiac chest pain), and when combined with antibiotics can be used to treat Helicobacter pylori eradication. The goals of GERD management are threefold: prompt and sustained symptom control, healing of the injured esophageal mucosa and prevention of GERD-related complications (including stricture formation, Barrett's esophagus, and/or adenocarcinoma). Pharmacological therapy with proton pump inhibitors forms the basis of both acute and long-term management of GERD. Proton pump inhibitors provide effective relief of symptoms and healing of the esophagitis, as well as sustaining long-term remission.

Although therapeutic efficacy is the primary concern for a therapeutic agent, the solid-state form, as well as the salt form, and the properties unique to the particular form of a drug candidate are often equally important to its development. Each solid state form (crystalline or amorphous) of a drug candidate can have different physical and chemical properties, for example, solubility, stability, or the ability to be reproduced. These properties can impact the ultimate pharmaceutical dosage form, the optimization of manufacturing processes, and absorption in the body. Moreover, finding the most adequate solid form for further drug development can reduce the cost of that development.

The chirality of a drug molecule can also be important Chiral molecules, as is well known to chemists, exist in two enantiomorphic forms that are mirror images of each other. In the same manner that left and right hands are mirror images of each other and cannot be superimposed over each other, enantiomers of chiral molecules cannot be superimposed over each other. The only difference in the molecules is their orientation in three dimensional space. The physical properties of enantiomers are identical to each other with the exception of the rotation of the plane of polarized light. It is this rotation of polarized light that allows one skilled in the art to determine if a chiral material is enantiomerically pure. In biological systems, however, different enantiomers can have very different effects. For example, a pure enantiomer may be used as the active pharmaceutical ingredient (API) because only one enantiomer may have the desired biological activity or the opposite enantiomer may produce unwanted side effects. Alternatively, one enantiomer may be eliminated from the body more rapidly than the other. One example of a drug that is a pure enantiomer is thalidomide.

The only difference in the physical properties of the two enantiomers of a chiral compound on a molecular level is the optical rotation of the molecule. All the properties associated with the solid-state, the supramolecular properties of the materials are the same. In other words, two enantiomers show the same polymorphism behavior. Accordingly, the melting point, vibrational spectra, X-ray diffraction patterns are the same for the same crystal form of the two enantiomers. Therefore, in general, solid-state analytical methods are not useful for the detection of the chiral purity of a given material. Methods that are sensitive to the optical activity are usually performed from a solution of the material of interest, e.g. optical rotation (Polarimetry) and/or chiral HPLC analysis.

Optical rotation occurs because optically active samples have different refractive indices for left- and right-circularly polarized light, i.e. left- and right-circularly polarized light travel through an optically active sample at different velocities. This condition occurs because the chiral center has a specific geometric arrangement of four different substituents, each of which has a different electronic polarizability. Light travels trough matter by interacting with the electron clouds that are present. Left-circularly polarized light therefore interacts with an anisotropic medium differently than does right-circularly polarized light. Linearly or plane-polarized light is the superposition of equal intensities of left- and right-circularly polarized light. As plane-polarized light travels through an optically active sample, the left- and right-circularly polarized components travel at different velocities. This difference in velocities creates a phase shift between the two circularly polarized components when they exit the sample.

Obtaining substantially pure crystalline or amorphous (or non-crystalline) forms is extremely useful in drug development. It permits better characterization of the drug candidate's chemical and physical properties and thereby allows identification of the form or forms with the desired combination of therapeutic effect and comparative ease of manufacture. The solid state form may possess more favorable pharmacology than the amorphous form or may be easier to process. It may also possess greater storage stability.

The solid state physical properties of a drug candidate may also influence its selection as a pharmaceutical active ingredient and the choice of form for its pharmaceutical composition. One such physical property, for example, is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate. Another important solid state property of a pharmaceutical compound is its dissolution rate in aqueous fluid. The rate of dissolution of an active ingredient in a patient's gastrointestinal fluid may have therapeutic consequences since it impacts the rate at which an orally-administered active ingredient may reach the patient's bloodstream.

These practical physical properties are influenced by the properties of the particular solid state form of the compound, for example, by the conformation and orientation of molecules in the unit cell of the crystalline compound. A crystalline form often has thermal behavior characteristics different from the amorphous form or another polymorphic form. Thermal behavior is measured in the laboratory by such techniques as capillary melting point, thermogravimetric analysis (TG) and differential scanning calorimetry (DSC) and may be used, for example, to distinguish some polymorphic forms from others. A particular solid state form generally possesses distinct crystallographic and spectroscopic properties detectable by powder X-ray diffraction (XRPD), single crystal X-ray crystallography, and infrared spectrometry among other techniques.

### SUMMARY OF THE INVENTION

The invention relates to solid state forms of enantiopure ilaprazole, 2[[(4-methoxy-3-methyl-2-pyridinyl)-methyl]sulfinyl]-5-(1H-pyrrol-1-yl) 1H-Benzimidazole. The invention also relates to a pharmaceutical composition for inhibiting gastric acid secretion comprising a solid form of ilaprazole according to the invention in an amount effective to inhibit gastric acid secretion and a pharmaceutically acceptable carrier. The invention also provides methods of treatment for various acid-related gastrointestinal (GI) disorders such as those discussed above.

### BRIEF DESCRIPTION OF THE DRAWINGS

FIG. 1 shows the XRPD pattern for ilaprazole(+), Form A.

FIG. 2 is the DSC thermogram of ilaprazole(+), Form A.

FIG. 3 is the solid state ¹³C CP/MAS NMR of ilaprazole(+), Form A.

FIG. 4 is the IR spectrum of ilaprazole(+), Form A.

FIG. 5 is the Raman spectrum of ilaprazole(+), Form A.

FIG. 6 is the XRPD pattern for ilaprazole(−), Form O.

FIG. 7 is the DSC thermogram of ilaprazole(−), Form O.

FIG. 8 is the solid state ¹³C CP/MAS NMR of ilaprazole(−), Form O.

FIG. 9 is the IR spectrum of ilaprazole(−), Form O.

FIG. 10 is the Raman spectrum of ilaprazole(−), Form O.

FIG. 11 is the XRPD pattern for amorphous ilaprazole(−).

FIG. 12 is an ORTEP drawing of ilaprazole(−), Form A. Atoms are represented by 50% probability anisotropic thermal ellipsoids.

FIG. 13 is a packing diagram of ilaprazole(−), Form A viewed down the crystallographic a axis.

FIG. 14 is a packing diagram of ilaprazole(−), Form A viewed down the crystallographic b axis.

FIG. 15 is a packing diagram of ilaprazole(−), Form A viewed down the crystallographic c axis.

FIG. 16 is the calculated XRPD pattern of ilaprazole(−), Form A.

FIG. 17 is the experimental XRPD pattern of ilaprazole(−), Form A.

FIG. 18 is a comparison of the calculated XRPD pattern of ilaprazole(−), Form A to the experimental XRPD pattern of ilaprazole(−), Form A.

FIG. 19 is a representative tableting process for a delayed release pharmaceutical composition of the invention.

### DETAILED DESCRIPTION OF THE INVENTION

### 1. Enantiopure Solid State Forms of Ilaprazole

Ilaprazole, 2[[(4-methoxy-3-methyl-2-pyridinyl)-methyl]sulfinyl]-5-(1H-pyrrol-1-yl) 1H-Benzimidazole, is a substituted benzimidazole that acts as a proton pump inhibitor. Ilaprazole selectively and irreversibly inhibits gastric acid secretion through inhibition of the hydrogen-potassium adenosine triphosphatase (H+K+-ATPase) (proton pump) mechanism. Inhibition of the proton pump occurs by formation of disulfide covalent bonds with accessible cysteines on the enzyme. Ilaprazole has a prolonged duration of action that persists after their elimination from plasma. See, for example, U.S. Pat. Nos. 5,703,097 and 6,280,773, which are incorporated herein by reference.

Ilaprazole has the empirical formula C₁₉H₁₈N₄O₂S having a molecular weight of 366.44 daltons. Ilaprazole is a chiral molecule and has the following structural formula (I):

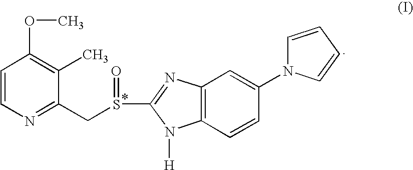

Ilaprazole possesses a chiral sulfur atom, S*. This can be depicted as follows with the lone pair of electrons on the chiral sulfur atom occupying one position in each stereoisomer, as shown below:

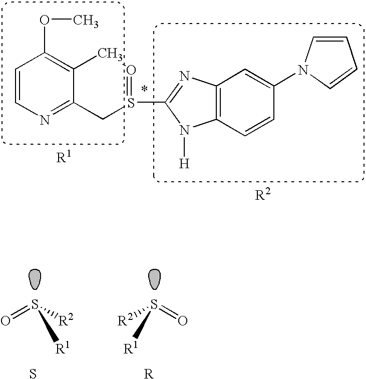

The absolute structure and absolute confirmation of (−)-S-ilaprazole was made through single crystal structure determination and is shown below. See Example 7.

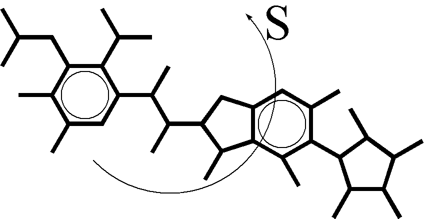

Thus, its complimentary enantiomer is (+)-R-ilaprazole, as shown below.

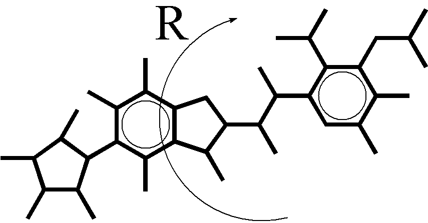

Separation of the enantiomers in a racemic mixture can be accomplished by their interaction (chemical or physical) with optically active reagents. One of the most common methods today is chiral chromatography, in which an optically active compound is immobilized on the stationary phase. The differences in interaction between the solid phase and the enantiomers is sufficiently different to allow separation. This separation allows the enantiomers to be purified and/or quantitated.

A particularly useful type of chiral chromatography is a chiral HPLC which requires chiral HPLC columns. Chiral HPLC columns can be prepared by immobilizing single enantiomers onto the stationary phase. For instance, a CHIRALPACK AS-H, 3 cm i.d. column may be used under the following conditions: mobile phase: hexane/ethanol/DEA-70/30/0.1%; Flow rate: 40 ml/min; and Feed concentration: 7.5 g/L.

Resolution relies on the formation of transient stereoisomers on the surface of the column packing. The compound which forms the most stable stereoisomer will be most retained, whereas the opposite enantiomer will form a less stable stereoisomer and will elute first. As understood by those of skill in the art, to achieve discrimination between enantiomers, i.e. chiral recognition, there must be a minimum of three points of interaction.

The forces that lead to this interaction are very weak and require careful optimization by adjustment of the mobile phase and temperature to maximize selectivity. Chromatography is a multi-step method where the separation is a result of the sum of a large number of interactions. The intermolecular forces involved with chiral recognition are polar/ionic interactions, pi-pi interactions, hydrophobic effects and hydrogen bonding. These can be augmented by the formation of inclusion complexes and binding to specific sites such as peptide or receptor sites in complex phases.

In the solid state, pure enantiomers can be very different from the racemic material. This is particularly true in the crystalline form. Racemates can crystallize as a conglomerate (where the two enantiomers form identical, mirror-image crystals that are the pure enantiomer), a racemic compound (where the two enantiomers coexist and are incorporated into specific locations of the crystal) or a solid solution (where the enantiomers can be located at any point within the crystal). Since enantiomerically pure materials (also known as enantiopure materials) are, by definition, missing one of the enantiomers, crystal forms can be considerably different in a racemic compound. Solid state forms can be characterized by various physical properties such as solubility, melting point, x-ray powder diffraction, solid state NMR, Raman, and IR spectroscopy. These properties can be considerably different between an enantiomer and the racemic material, however, the properties are not different between the two enantiomers.

This invention relates to solid state forms of enantiopure ilaprazole, that is the solid state form of one member of an enantiomeric pair. More particularly, the invention relates to two polymorphic forms, A and O, of enantiopure ilaprazole and the amorphous form of enantiopure ilaprazole. As discussed above, each member of a pair of enantiomers has physical properties that are identical to each other with the exception of the rotation of the plane of polarized light. The enantiopure forms of ilaprazole described in the examples below are crystalline ilaprazole(−), Form A; crystalline ilaprazole(+), Form A; crystalline ilaprazole(−), Form O; and amorphous ilaprazole(−).

In using the term “enantiopure,” or an “enantiopure form,” it is meant that one enantiomer is predominately present. While minor amounts of the other enantiomeric forms may be present, the desired enantiomer should constitute at least 90% of all forms of the compound. For example, enantiopure ilaprazole(+) should be 90% or more ilaprazole(+), containing less than 10% of other enantiomeric forms of ilaprazole. Preferably, the enantiopure form constitutes at least 95% of the desired enantiomer, more preferably at least 98%, and most preferably at least 99%.

The two polymorphic forms of enantiopure ilaprazole have been identified and are labeled Form A and Form O. These forms can be identified in the solid state by x-ray powder diffraction (XRPD) and solid state NMR, infra-red (IR) or Raman spectroscopy. Characteristic peaks from each technique are listed in the tables below. Although the forms listed are identified as a particular enantiomer, the peaks are characteristic of the solid state form and independent of the enantiomer. Both forms are available to either enantiomer. The particular enantiomers were identified by chiral HPLC and the absolute configuration for ilaprazole(−), Form A was determined by single crystal x-ray diffraction (as shown in the figures).

Tables 1-3 below report the characteristic peaks in the XRPD patterns, IR spectra, and Raman spectra, respectively, for Forms A and O. The XRPD peaks are reported, here and in the examples, as ±0.2°2θ. Similarly, the IR and Raman peaks are reported as ±4 cm⁻¹. Additional data for each form which may be used to identify each form is presented in the Examples below. Each form disclosed here possesses advantages vis-à-vis the other forms, for example, for a particular formulation or processing. Tables 1-3, and the examples below, report the data for the particular enantiomer studied although, as discussed above, these physical properties are the same for both enantiomers of each form.

TABLE 1
Characteristic XRPD Peaks for Enantiopure Ilaprazole Forms

| | |
|---|---|
| | Peaks Positions |
| Form | (°2θ ± 0.2 °2θ) |

| | | |
|---|---|---|
| A(+) | 8.5 | 13.1 |
| O(−) | 11.5 | 12.2 |

TABLE 2
Characteristic IR Peaks for Enantiopure Ilaprazole Forms

| | |
|---|---|
| | Peaks Positions |
| Form | (cm⁻¹ ± 1 cm⁻¹) |

| | | |
|---|---|---|
| A(+) | 712 | 776 |
| O(−) | 837 | 885 |

TABLE 3
Characteristic RAMAN Peaks for Enantiopure Ilaprazole Forms

| | |
|---|---|
| | Peaks Positions |
| Form | (cm⁻¹ ± 1 cm⁻¹) |

| | | |
|---|---|---|
| A(+) | 448 | 625 |
| O(−) | 444 | 642 |

### 2. Pharmaceutical Compositions and Methods

Ilaprazole is useful for inhibiting gastric acid secretion as well as for providing gastrointestinal cytoprotective effects in mammals, including humans. In a more general sense, ilaprazole may be used for prevention and treatment of gastrointestinal inflammatory diseases in mammals, including e.g. gastritis, gastric ulcer, and duodenal ulcer. As discussed above, such GI disorders include, for example, gastroesophageal reflux disease (GERD), peptic ulcer disease, Zollinger-Ellison Syndrome (ZES), ulcers, and nonsteroidal anti-inflammatory drug (NSAID)-induced gastropathy. Ilaprazole may furthermore be used for prevention and treatment of other gastrointestinal disorders where cytoprotective and/or gastric antisecretory effect is desirable, e.g. in patients with gastrinomas, in patients with acute upper gastrointestinal bleeding, and in patients with a history of chronic and excessive alcohol consumption.

The results of Phase 1 clinical studies conducted with ilaprazole suggest that at the doses studied, suppression of gastric acid occurs over a 24-hour period. In Phase 2 clinical studies conducted with ilaprazole, the results indicated that ilaprazole at the doses studied provided symptomatic relief for patients with gastric-acid related disorders and promoted rapid healing of acid-related gastric and duodenal ulcers.

Accordingly, the invention relates to a pharmaceutical composition for inhibiting gastric acid secretion comprising a solid state form of enantiopure ilaprazole according to the invention in an amount effective to inhibit gastric acid secretion and a pharmaceutically acceptable carrier. Pharmaceutical compositions are discussed below.

The invention also relates to the treatment of various acid-related gastrointestinal (GI) inflammatory diseases and disorders such as those discussed above and providing gastrointestinal cytoprotection. The invention provides a method for inhibiting gastric acid secretion by administering to mammals a solid state form of enantiopure ilaprazole according to the invention, or a pharmaceutical composition containing it, in an amount sufficient to inhibit gastric acid secretion. The invention also provides a method for the treatment of gastrointestinal inflammatory diseases in mammals by administering to mammals a solid state form of enantiopure ilaprazole according to the invention, or a pharmaceutical composition containing it, in an amount sufficient to treat gastrointestinal inflammatory disease. The invention further provides a method for providing gastrointestinal cytoprotective effects in mammals by administering to mammals a solid state form of enantiopure ilaprazole according to the invention, or a pharmaceutical composition containing it, in an amount sufficient to provide gastrointestinal cytoprotective effects.

The invention relates to pharmaceutical compositions comprising a therapeutically effective amount of a solid state form of enantiopure ilaprazole of the invention and a pharmaceutically acceptable carrier, (also known as a pharmaceutically acceptable excipient). As discussed above, the solid state forms of enantiopure ilaprazole are useful for the treatment of various acid-related gastrointestinal (GI) disorders. Pharmaceutical compositions for the treatment of those diseases and disorders contain a therapeutically effective amount of a solid state form of enantiopure ilaprazole of the invention to inhibit gastric secretion as appropriate for treatment of a patient with the particular disease or disorder.

A “therapeutically effective amount of a solid state form of enantiopure ilaprazole to inhibit gastric secretion” (discussed here concerning the pharmaceutical compositions) refers to an amount sufficient to inhibit or reduce gastric secretion and thereby to treat, i.e. to reduce the effects, inhibit or prevent, various acid-related gastrointestinal (GI) disorders and/or provide gastrointestinal cytoprotection. The actual amount of crystalline form of racemic ilaprazole required for treatment of any particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of the solid state form of enantiopure ilaprazole according to the invention; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference.

The absorption of the solid state forms of enantiopure ilaprazole can be altered depending on when the subject consumes food in relation to when the dosage is administered. The rate of absorption can also depend on the type of diet consumed, particularly if the diet has a high concentration of fats. These factors, as well as others known to those of skill in the art that can affect the absorption of proton pump inhibitors, can consequently influence the efficacy of the solid state forms of enantiopure ilaprazole in inhibiting gastric acid secretion. It has been found that the absorption of the solid state forms of enantiopure ilaprazole can be delayed and the bioavailability increased when administered in the fed state or approximately five minutes before a high-fat meal, compared to administration in the fasted state. Administration of the solid state forms of enantiopure ilaprazole approximately one hour before a high-fat meal produces results similar to that observed during administration in the fasted state. These findings are consistent with similar studies performed with other tableted formulations of proton pump inhibitors.

A pharmaceutical composition of the invention may be any pharmaceutical form which contains and retains the solid state form of enantiopure ilaprazole according to the invention. The pharmaceutical composition may be, for example, a tablet, capsule, liquid suspension, injectable, topical, or transdermal. A comprehensive disclosure of suitable formulations may be found in U.S. Published Application No. 2006/013868, herein incorporated by reference in its entirety. For injectables and liquid suspensions, those should be formulated such that the solid state form of enantiopure ilaprazole is present in the formulated composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is one having a solid state form of enantiopure ilaprazole of the invention, a carrier should be chosen that maintains the solid state form of enantiopure ilaprazole of the invention. In other words, the carrier should not substantially alter the crystalline form of the enantiopure ilaprazole of the invention. Nor should the carrier be otherwise incompatible with a solid state form of enantiopure ilaprazole according to the invention, such as by producing any undesirable biological effect or otherwise interacting in a deleterious manner with any other component(s) of the pharmaceutical composition.

The pharmaceutical compositions of the invention are preferably formulated in unit dosage form for ease of administration and uniformity of dosage. A “unit dosage form” refers to a physically discrete unit of therapeutic agent appropriate for the patient to be treated. It will be understood, however, that the total daily dosage of a solid state form of enantiopure ilaprazole of the invention and its pharmaceutical compositions according to the invention will be decided by the attending physician within the scope of sound medical judgment.

It may be desirable to administer the dosage in a composition where the solid state form of enantiopure ilaprazole is released from the dosage form as a first and a second dose where each of the first and second dose contain a sufficient amount of the solid state form of enantiopure ilaprazole to raise plasma levels to a desired concentration. Suitable formulations to achieve this are disclosed in PCT Published Application No. WO 2006/009602, herein incorporated by reference in its entirety.

Because the solid state forms of enantiopure ilaprazole of the invention are more easily maintained during preparation, solid dosage forms are preferred for the pharmaceutical composition of the invention. Solid dosage forms for oral administration, which includes capsules, tablets, pills, powders, and granules, are particularly preferred. In such solid dosage forms, the active compound is mixed with at least one inert, pharmaceutically acceptable carrier (also known as a pharmaceutically acceptable excipient). The solid dosage form may, for example, include one or more pharmaceutical carriers/excipients as known in the art, including; a) fillers or extenders such as starches, lactose, lactose monohydrate, sucrose, glucose, mannitol, sodium citrate, dicalcium phosphate, and silicic acid; b) binders such as, for example, carboxymethylcellulose, microcrystalline cellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, sodium starch glycolate, and sodium carbonate; e) dissolution retarding agents such as paraffin; f) absorption accelerators such as quaternary ammonium compounds; g) wetting agents such as, for example, cetyl alcohol and glycerol monostearate; h) absorbents such as kaolin and bentonite clay, i) lubricants such as talc, calcium stearate, magnesium stearate, magnesium hydroxide, solid polyethylene glycols, sodium lauryl sulfate; and j) glidants such as colloidal silicon dioxide. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980), which is hereby incorporated by reference in its entirety, discloses various carriers used in formulating pharmaceutical compositions and known techniques for the preparation thereof. Solid dosage forms of pharmaceutical compositions of the invention can also be prepared with coatings and shells such as enteric coatings and other coatings well known in the pharmaceutical formulating art, including formulations and coatings designed to provide for extended release of the active pharmaceutical ingredient (API). For example, U.S. Pat. No. 6,605,303, incorporated herein by reference, describes oral extended release formulations for the proton pump inhibitor omeprazole. Accordingly, the solid dosage form may be an extended or delayed release formulation. An exemplary delayed-release tablet formulation is described in Example 8, below.

A solid state form of enantiopure ilaprazole of the invention can also be in a solid micro-encapsulated form with one or more carriers as discussed above. Microencapsulated forms of a solid state form of enantiopure ilaprazole of the invention may also be used in soft and hard-filled gelatin capsules with carriers such as lactose or milk sugar as well as high molecular weight polyethylene glycols and the like.

The invention also provides methods for the treatment of the GI disorders discussed above. The solid forms of enantiopure ilaprazole and pharmaceutical compositions containing them may, according to the invention, be administered using any amount, any form of pharmaceutical composition and any route of administration effective for the treatment. After formulation with an appropriate pharmaceutically acceptable carrier in a desired dosage, as known by those of skill in the art, the pharmaceutical compositions of this invention can be administered to humans and other animals orally, rectally, parenterally, intraveneously, intracisternally, intravaginally, intraperitoneally, topically (as by powders, ointments, or drops), bucally, as an oral or nasal spray, or the like, depending on the location and severity of the condition being treated. As discussed above, when administering a pharmaceutical compositions of the invention via one of these routes, the pharmaceutical composition contains the solid form of enantiopure ilaprazole in one of the crystalline forms of the invention. Oral administration using tablets or capsules is generally preferred.

In certain embodiments, the solid forms of enantiopure ilaprazole according to the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. For extended release formulations, the dosage may range from about 5 mg to about 80 mg, preferably ranging from about 10 mg to about 50 mg ilaprazole, and more preferably ranging from about 20 mg to about 40 mg.

### EXAMPLES

Example 1 describes the preparation of ilaprazole. Examples 2-5 describe the preparation and characterization of four solid state forms of ilaprazole(+), Form A; ilaprazole(−), Form A; ilaprazole(−), Form O; and amorphous ilaprazole(−). The solid state forms were characterized by various techniques. Each technique is described below. Table 4 shows the particular enantiopure solid state form and the techniques used to characterize that form. Example 6 describes solubility studies of ilaprazole, and example 7 describes single crystal preparation.

TABLE 4
Characterization Techniques for Enantiopure Ilaprazole Forms

| Form | Methods | Observations |
|---|---|---|
| A | XRPD | Form A(+) |
| | DSC | Form A(−) |
| | | Endotherm onset |
| | | 169 |
| | | (max 173) |
| | ¹³C CP/MAS ssNMR | Form A(+) |
| | IR | Form A(+) |
| | Raman | Form A(+) |
| O | XRPD | Form O(−) |
| | DSC | Form O(−): |
| | | Endotherm onset |
| | | 172 |
| | | (max 175) |
| | ¹³C CP/MAS ssNMR | Form O(−) |
| | IR | Form O(−) |
| | Raman | Form O(−) |
| Amorphous | XRPD | Amorphous(−) |

Differential Scanning Calorimetry (DSC): Analyses were carried out on a TA Instruments differential scanning calorimeter 2920 or Q1000 The instrument was calibrated using indium as the reference material. The sample was placed into an aluminum, non-crimped DSC pan and the weight accurately recorded. The sample cell was equilibrated at 25° C. and heated under a nitrogen purge at a rate of 10° C./min, up to a final temperature of 250 or 350° C.

IR Spectroscopy: Infrared spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. An attenuated total reflectance (ATR) accessory (Thunderdome™, Thermo Spectra-Tech), with a germanium (Ge) crystal was used for data acquisition. The spectra represent 256 co-added scans collected at a spectral resolution of 4 cm⁻¹. A background data set was acquired with a clean Ge crystal. Log 1/R (R=reflectance) spectra were acquired by taking a ratio of these two data sets against each other. Wavelength calibration was performed using polystyrene.

Solid State ¹³C CP/MAS NMR Analyses (ssNMR): Samples were prepared for solid-state NMR spectroscopy by packing them into 4 mm PENCIL type zirconia rotors. The spectra were acquired on an INOVA-400 spectrometer using ¹H cross-polarization (CP) and magic angle spinning, (MAS). The specific acquisition parameters are listed in Table 5:

TABLE 5
¹³C ssNMR Acquisition Parameters

| Reference: | Glycine (external reference at 176.5 ppm) |
|---|---|
| Temperature: | Ambient |
| Pulse sequence: | xpolvtlrho1 |
| Relaxation delay: | 10 seconds |
| Pulse width: | 2.2 μseconds |
| Acquisition time: | 0.030 seconds |
| Spectral width: | 44994.4 Hz, (447.517 ppm) |
| Acquired points: | 32000 |
| ¹H Decoupling | 400 MHz |
| SPINAL-64 decoupling | |
| Cross Polarization tangent | |
| RAMP-CP on C13 | |
| Contact Time: | 5.0 mseconds |
| Spin rate: | 12000 Hz |
| Data processing: | |
| Backward linear prediction: | 3 points |
| Line broadening: | 10.0 Hz |
| FT size: | 131072 |

Raman Spectroscopy: FT-Raman spectra were acquired on an FT-Raman 960 spectrometer (Thermo Nicolet). This spectrometer uses an excitation wavelength of 1064 nm. Approximately 0.5 W of Nd:YVO4 laser power was used to irradiate the sample. The Raman spectra were measured with an indium gallium arsenide (InGaAs) detector. The samples were prepared for analysis by placing the sample into a capillary. A total of 256 sample scans were collected from 3600-100 cm⁻¹ at a spectral resolution of 4 cm⁻¹, using Happ-Genzel apodization. Wavelength calibration was performed using sulfur and cyclohexane.

X-ray Powder Diffraction (XRPD): XRPD patterns were obtained using an Inel XRG-3000 Diffractometer that was equipped with a curved position-sensitive detector with a 2θ range of 120°. Real time data were collected using Cu Kα radiation starting at approximately 4° 2θ at a resolution of 0.03° 2θ. The tube voltage and amperage were set to 40 kV and 30 mA, respectively. Samples were run for 5 or 15 minutes. Patterns are displayed from 2.5 to 40° 2θ to facilitate direct pattern comparisons. Samples were prepared for analysis by packing them into thin-walled glass capillaries. Each capillary was mounted onto a goniometer head that is motorized to permit spinning of the capillary during data acquisition. Instrument calibration was performed daily using a silicon reference standard.

XRPD Peak Picking Methods: Any XRPD files generated from an Inel instrument were converted to Shimadzu .raw file using File Monkey version 3.0.4. The Shimadzu raw file was processed by the Shimadzu XRD-6000 version 4.1 software to automatically find peak positions. The “peak position” means the maximum intensity of a peaked intensity profile. Parameters used in peak selection are shown with each parameter set of the data. The following processes were used with the Shimadzu XRD-6000 “Basic Process” version 2.6 algorithm: 1) smoothing was done on all patterns; 2) the background was subtracted to find the net, relative intensity of the peaks; and 3) the Cu K alpha2 (1.5444 Å wavelength) peak was subtracted from the pattern at 50% of the Cu K alpha1 (1.5406 Å) peak intensity for all patterns.

Each figure listing XRPD peaks for each form shows peaks selected by the peak picking method described above. Tables listing peaks for each form shows peaks that are visually present in the diffractogram. The peak positions in bold denote the characteristic peak set. I/Iₒ is relative intensity.

### Example 1

### Separation of Ilaprazole into Ilaprazole(+) and Ilaprazole(−)

The racemic mixture was purified into enantiomers using preparative chiral chromatography, such as that discussed above. The mobile phase was water:acetonitrile:triethyamine. Triethylamine was used to stabilize the ilaprazole in solution. The fractions were collected that contained the separate enantiomers. The enantiomers were confirmed by NMR, optical rotation and analytical chiral chromatography. The (+) and (−) rotations were associated to the R and S configurations and the two enantiomers were assigned as R(+) (peak 1) and S(−) (peak 2).

Each ilaprazole enantiomer was then purified and crystallized as follows: Each enantiomer sample (20 g, 1.0 part) was dissolved in a mixture of methylene chloride (900 g, 45 parts), and triethylamine (10 g, 0.50 part), and water (300 g, 15 parts). After layer separation, the organic layer was concentrated to ca. 200 mL (10 volumes) and subjected to silica gel column purification [silica gel: 200 g (10 parts); column pre-treated with 3% NH₄OH/MeCN to pH 10-11; eluted with 3% NH₄OH/MeCN]. The pure fractions were concentrated until distillation stopped; the resulting solid was co-distilled with 0.5% NH₄OH/EtOH (50 g, 2.5 parts). Methylene chloride (160 g, 8.0 parts) was charged and the resulting solution was concentrated at maximum 25° C. under reduced pressure to ca. 50 mL (2.5 volumes). 0.5% NH₄O/EtOH (40 g, 2.0 parts) was charged and the contents were concentrated at maximum temperature of 25° C. under reduced pressure to ca. 40 mL (2.0 volumes). 0.5% NH₄OH/EtOH (10 g, 0.50 part) was charged and the contents were adjusted to 5° C. (2-8° C.) and agitated for 30 minutes. The slurry was filtered and rinsed with 3% NH₄OH/EtOH (20 g, 1.0 part, pre-cooled to 5° C.), EtOH (20 g, 1.0 part, pre-cooled to 5° C.) and MTBE (40 g, 1.0 part, pre-cooled to 5° C.). The filter cake was dried under vacuum at maximum 50° C.

A summary of the yield and purity of the crystallized ilaprazole enantiomers is set forth below in Table 6.

TABLE 6
Yield and purity of the crystallized Ilaprazole enantiomers

| | | | Purity | Purity | Color |
|---|---|---|---|---|---|
| | | | (HPLC | (HPLC | of the |
| Enantiomer | Scale (g) | Yield (g/%) | A %) | wt %) | product |
| Ilaprazole(−) | 20 | 14.6 (73) | 99.9 | 99.1 | Off-white |
| Ilaprazole(+) | 20 | 15.3 (77) | 99.9 | 98.5 | Off-white |

### Example 2

### Preparation and Characterization of Ilaprazole(+), Form A

Approximately 16 mg of ilaprazole(+) was dissolved in approximately 2 mL of dichloromethane and 18 μL triethylamine. The solution was filtered through a 0.2 μm nylon filter and approximately 3 mL of hexanes was added. The turbid solution was then filtered through a 0.2 μm nylon filter into a glass vial. Solid formed upon standing at ambient temperature over night.

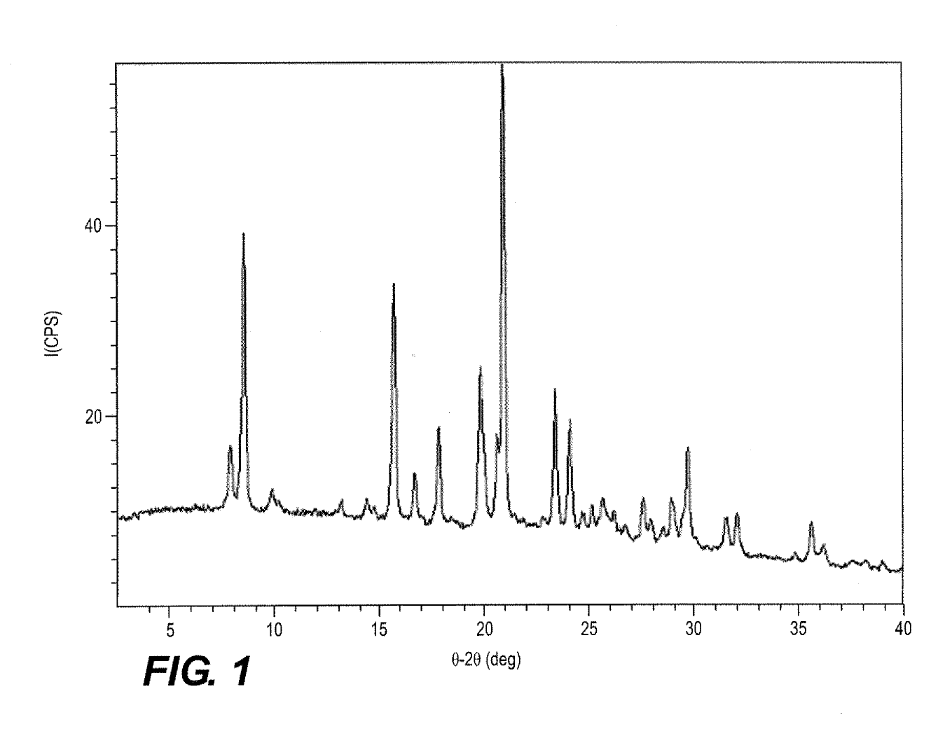

The XRPD pattern of Ilaprazole(+), Form A was obtained using an Inel XRG-3000 diffractometer. The measurement conditions are reported in Table 7. FIG. 1 shows the XRPD pattern for Ilaprazole(+), Form A. Table 8 reports twenty-six peaks identified in the XRPD pattern.

TABLE 7
Measurement Conditions for XRPD pattern of Ilaprazole(+), Form A
Measurement Condition:

| | |
|---|---|
| X-ray tube | |
| target = | Cu |
| voltage = | 40.0 (kV) |
| current = | 30.0 (mA) |
| Slits | |
| divergence slit = | 1.00000 (deg) |
| scatter slit = | 1.00000 (deg) |
| receiving slit = | 0.15000 (mm) |
| Scanning | |
| drive axis = | 2Theta/Theta |
| scan range = | 2.511-39.971 |
| scan mode = | Continuous Scan |
| scan speed = | 0.0040 (deg/min) |
| sampling pitch = | 0.0200 (deg) |
| preset time = | 300.00 (sec) |

Data Process Condition:

| | |
|---|---|
| Smoothing | [AUTO] |
| smoothing points = | 11 |
| B.G. Subtraction | [AUTO] |
| sampling points = | 11 |
| repeat times = | 30 |
| Ka1-a2 Separate | [MANUAL] |
| Ka1 a2 ratio = | 50.0 (%) |
| Peak Search | [AUTO] |
| differential points = | 9 |
| FWHM threshold = | 0.050 (deg) |
| intensity threshold = | 30 (par mil) |
| FWHM ratio (n − 1)/n = | 2 |
| System Error Correction: | [NO] |
| Precise Peak Correction: | [NO] |

TABLE 8
Peak Positions of Ilaprazole(+), Form A XRPD Pattern

| Peak | Position | | | |
|---|---|---|---|---|
| No. | (°2θ) | d-spacing | Intensity | I/Iₒ |
| 1 | 7.9 | 11.2 | 1281 | 13 |
| 3 | 9.9 | 9.0 | 446 | 5 |
| 2 | 8.5 | 10.4 | 5519 | 57 |
| 4 | 13.1 | 6.7 | 312 | 3 |
| 5 | 14.4 | 6.2 | 397 | 4 |
| 6 | 15.6 | 5.7 | 4814 | 50 |
| 7 | 16.6 | 5.3 | 956 | 10 |
| 8 | 17.8 | 5.0 | 2085 | 22 |
| 9 | 19.8 | 4.5 | 3351 | 35 |
| 10 | 20.6 | 4.3 | 1866 | 19 |
| 11 | 20.9 | 4.3 | 9671 | 100 |
| 12 | 23.3 | 3.8 | 2882 | 30 |
| 13 | 24.0 | 3.7 | 2272 | 23 |
| 14 | 24.7 | 3.6 | 323 | 3 |
| 15 | 25.1 | 3.5 | 483 | 5 |
| 16 | 25.7 | 3.5 | 679 | 7 |
| 17 | 26.1 | 3.4 | 476 | 5 |
| 18 | 27.5 | 3.2 | 876 | 9 |
| 19 | 27.9 | 3.2 | 435 | 4 |
| 20 | 28.9 | 3.1 | 901 | 9 |
| 21 | 29.4 | 3.0 | 558 | 6 |
| 22 | 29.7 | 3.0 | 2190 | 23 |
| 23 | 31.5 | 2.8 | 782 | 8 |
| 24 | 32.0 | 2.8 | 906 | 9 |
| 25 | 35.5 | 2.5 | 987 | 10 |
| 26 | 36.1 | 2.5 | 434 | 4 |

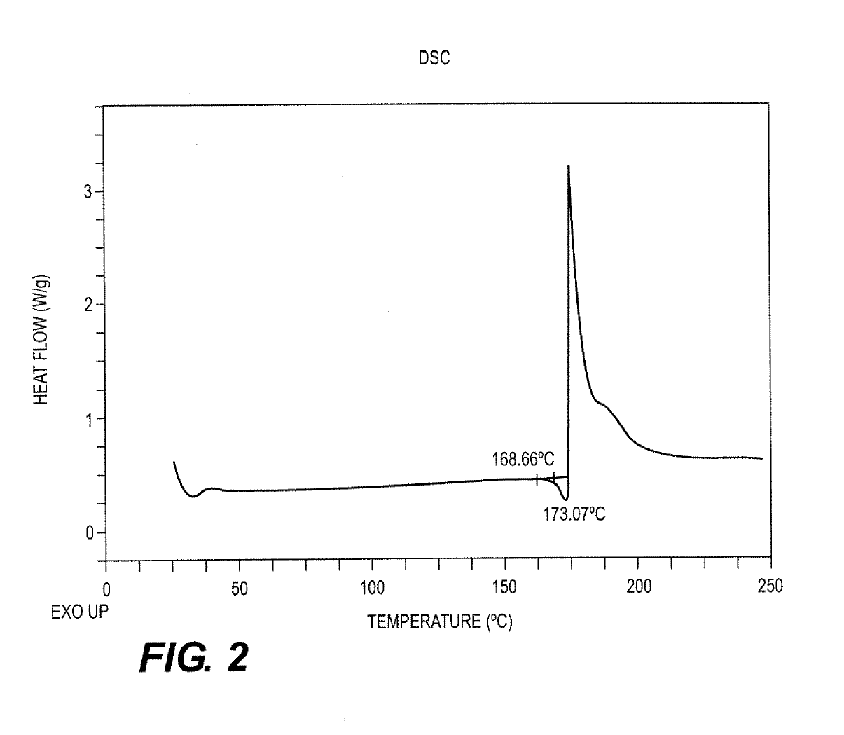

FIG. 2 is the solid state ¹³C CP/MAS NMR of ilaprazole(+), Form A, externally referenced against glycine at 176.5 ppm. Table 9 lists the ¹³C NMR peaks for ilaprazole(+),

TABLE 9
Solid state ¹³C NMR peaks of Ilaprazole(+), Form A

| δ ppm | Height |
|---|---|
| 163.9 | 126.6 |
| 154.7 | 95.0 |
| 149.3 | 131.2 |
| 148.4 | 101.6 |
| 141.9 | 122.3 |
| 138.9 | 104.7 |
| 137.4 | 104.6 |
| 123.6 | 98.6 |
| 122.1 | 133.5 |
| 120.3 | 97.0 |
| 119.0 | 119.8 |
| 110.8 | 49.9 |
| 109.1 | 97.6 |
| 107.2 | 112.2 |
| 61.1 | 106.5 |
| 56.2 | 141.8 |
| 12.5 | 138.3 |

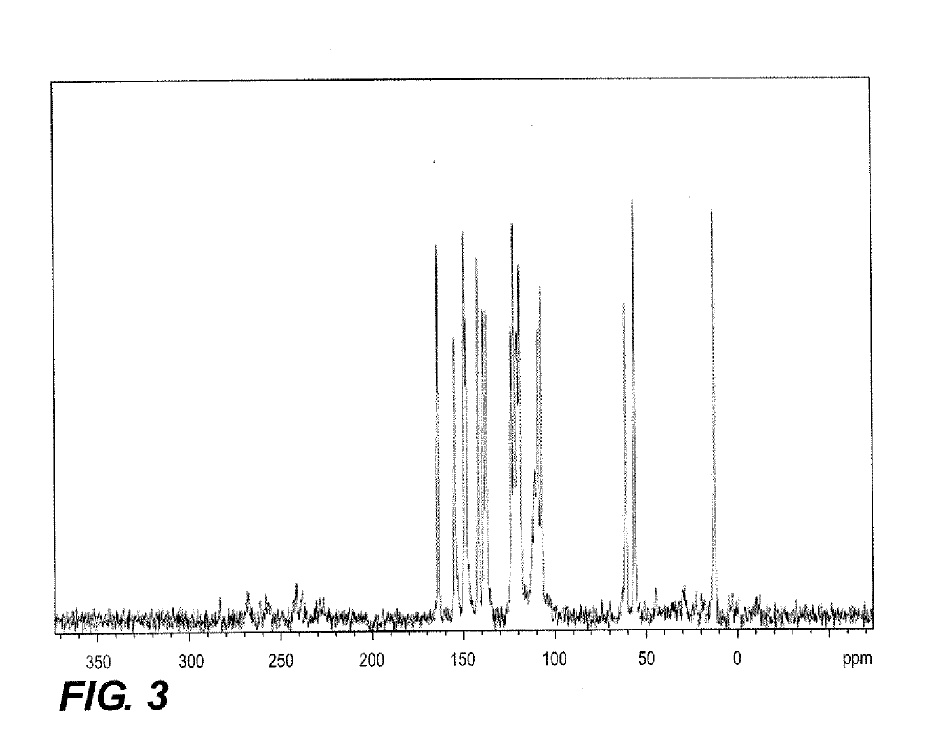

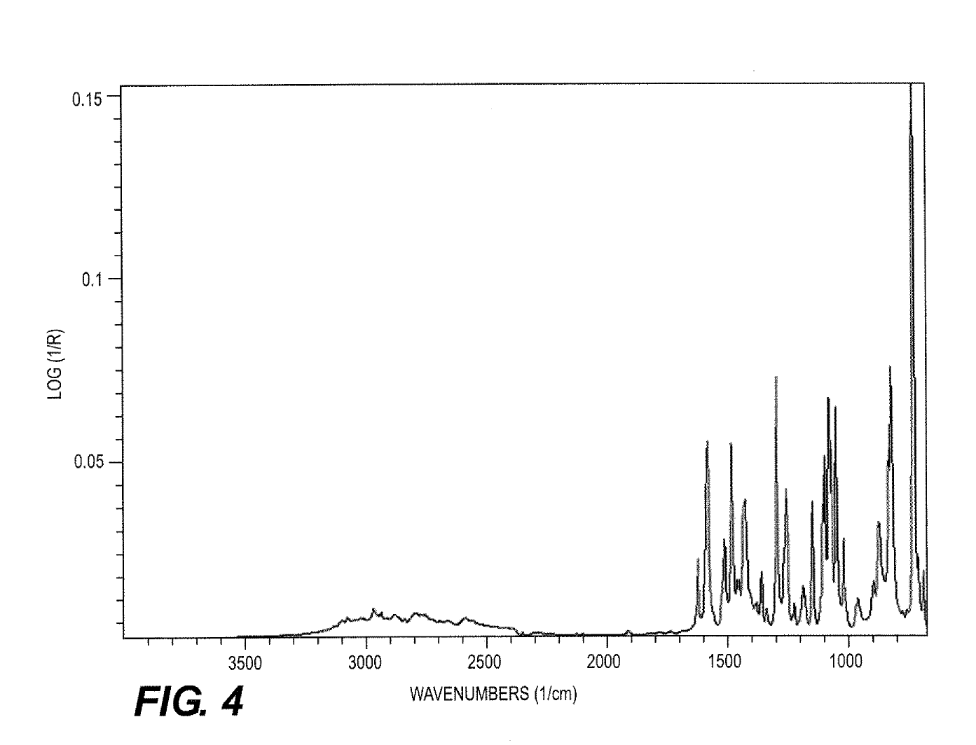

FIG. 3 is the DSC thermogram of Ilaprazole(+), Form A. The endotherm onset was 168° C. (max 173° C.). The endotherm is concurrent with an exotherm due to decomposition. FIG. 4 is the IR spectrum of ilaprazole(+), Form A. Table 10 lists the IR peaks.

TABLE 10
Peak Positions of Ilaprazole(+), Form A IR Spectrum

| | Intensity |
|---|---|
| Position | (Log |
| (cm⁻¹) | (1/R)) |
| 712 | 0.0244 |
| 730 | 0.156 |
| 758 | 0.0097 |
| 776 | 0.0094 |
| 822 | 0.076 |
| 833 | 0.0535 |
| 871 | 0.0333 |
| 875 | 0.0338 |
| 895 | 0.0177 |
| 960 | 0.0127 |
| 1019 | 0.0296 |
| 1049 | 0.0653 |
| 1068 | 0.0537 |
| 1079 | 0.0685 |
| 1097 | 0.0522 |
| 1104 | 0.0391 |
| 1148 | 0.0392 |
| 1186 | 0.0162 |
| 1223 | 0.0115 |
| 1256 | 0.0427 |
| 1295 | 0.0747 |
| 1337 | 0.0101 |
| 1359 | 0.0203 |
| 1379 | 0.0119 |
| 1424 | 0.04 |
| 1459 | 0.018 |
| 1480 | 0.0557 |
| 1510 | 0.0291 |
| 1581 | 0.0557 |
| 1622 | 0.0239 |
| 1732 | 0.0038 |
| 1910 | 0.004 |
| 2587 | 0.0078 |
| 2661 | 0.007 |
| 2794 | 0.0092 |
| 2839 | 0.0076 |
| 2879 | 0.0088 |
| 2935 | 0.0093 |
| 2967 | 0.0104 |
| 3021 | 0.0078 |
| 3074 | 0.0082 |
| 3098 | 0.0074 |

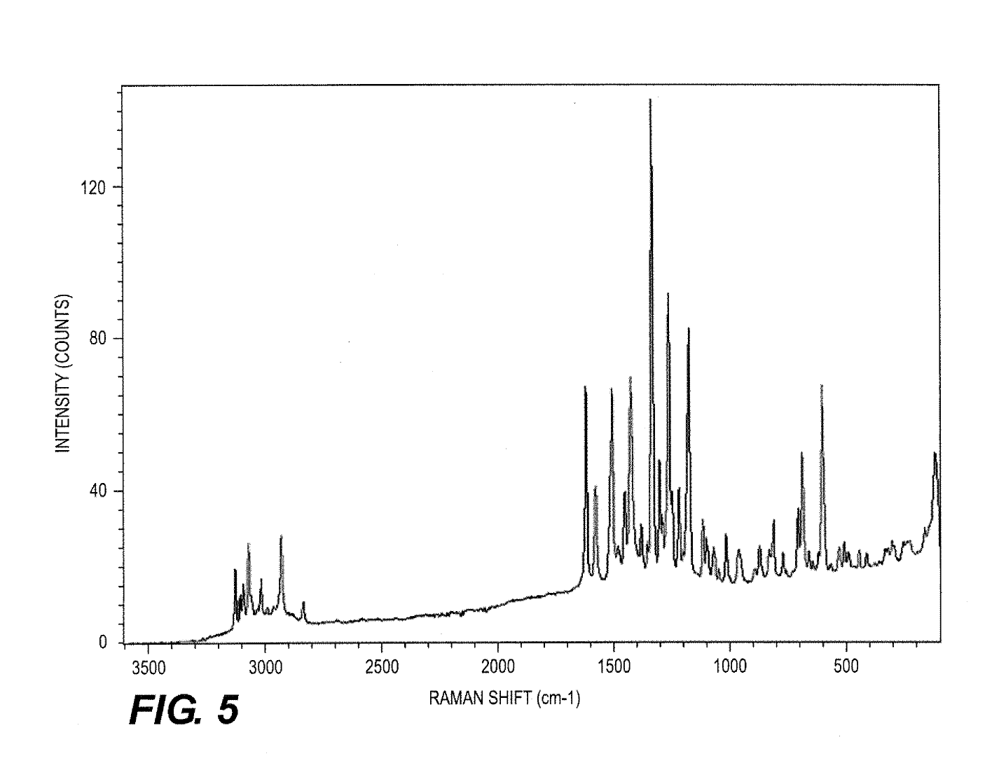

FIG. 5 is the Raman spectrum of ilaprazole(+), Form A. Table 11 lists the Raman peaks.

TABLE 11
Peak Positions of Ilaprazole(+), Form A, RAMAN Spectrum

| Position | |
|---|---|
| (cm-1)^{a} | Intensity |
| 418 | 3.424 |
| 448 | 4.57 |
| 496 | 4.257 |
| 513 | 6.855 |
| 534 | 6.027 |
| 571 | 1.753 |
| 600 | 29.865 |
| 608 | 50.183 |
| 625 | 5.091 |
| 648 | 2.742 |
| 664 | 5.672 |
| 694 | 31.552 |
| 712 | 17.604 |
| 762 | 1.159 |
| 777 | 5.943 |
| 816 | 14.597 |
| 836 | 7.037 |
| 876 | 8.295 |
| 896 | 2.476 |
| 967 | 8.892 |
| 1020 | 12.665 |
| 1053 | 3.197 |
| 1076 | 8.819 |
| 1104 | 10.708 |
| 1119 | 15.404 |
| 1180 | 65.514 |
| 1207 | 7.821 |
| 1223 | 24.147 |
| 1252 | 23.228 |
| 1266 | 75.791 |
| 1295 | 16.589 |
| 1307 | 32.656 |
| 1338 | 133.21 |
| 1359 | 10.874 |
| 1386 | 15.397 |
| 1430 | 54.474 |
| 1457 | 24.669 |
| 1485 | 10.391 |
| 1512 | 52.027 |
| 1583 | 26.673 |
| 1623 | 53.876 |
| 2839 | 6.091 |
| 2935 | 24.315 |
| 2967 | 6.094 |
| 2992 | 6.024 |
| 3022 | 13.912 |
| 3075 | 23.812 |
| 3099 | 13.44 |
| 3111 | 10.368 |
| 3131 | 18.205 |

### Example 3

### Preparation and Characterization of Ilaprazole(−), Form A

Approximately 20 mg of ilaprazole(−) was dissolved in 2 mL of THF and 50 μL triethylamine. The solution was then filtered through a 0.2 μm nylon filter into a glass vial containing ˜10 mL of cold hexanes (dry ice). The mixture was then kept in the dry ice bath for approximately 5 minutes. Yellow solid was collected by vacuum filtration followed by air dry for approximately 3 hours.

The XRPD pattern is crystalline and is nearly identical to the XRPD pattern of Ilaprazole(+), Form A as well as to that of racemic Form A. The XRPD peak positions are similar for all three patterns indicating the same crystalline form, although the relative intensities are different. The XRPD pattern obtained for Form A(−) also showed small peaks for O(−).

### Example 4

### Preparation and Characterization of Ilaprazole(−), Form O

Approximately 20 mg of ilaprazole(−) was dissolved in approximately 3 mL of THF and 10 μL of triethylamine. The solution was then filtered through a 0.2 μm nylon filter into a glass vial. Solids formed upon evaporation of the solvents at ambient within 24 hours.

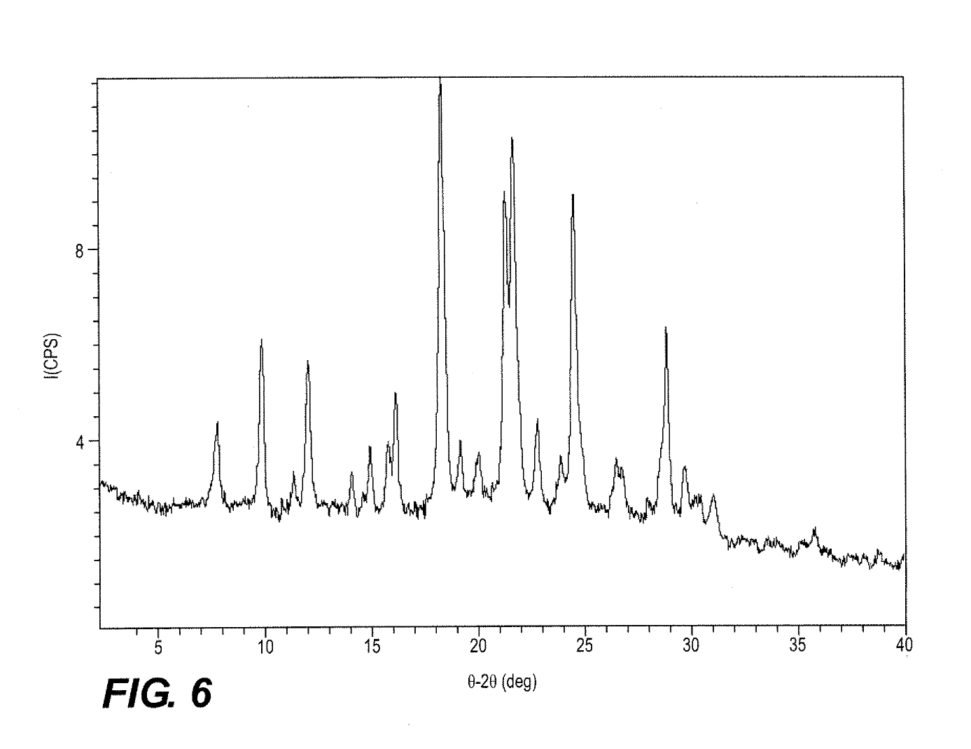

The XRPD pattern of Ilaprazole(−), Form 0 was obtained using an Inel YRG-3000 diffractometer. The measurement conditions are reported in Table 12. FIG. 6 shows the XRPD pattern for Ilaprazole(−), Form O. Table 13 reports 31 peaks identified in the XRPD pattern.

TABLE 12
Measurement Conditions for XRPD pattern of Ilaprazole(−), Form O.
Measurement Condition:

| | |
|---|---|
| X-ray tube | |
| target = | Cu |
| voltage = | 40.0 (kV) |
| current = | 30.0 (mA) |
| Slits | |
| divergence slit = | 1.00000 (deg) |
| scatter slit = | 1.00000 (deg) |
| receiving slit = | 0.15000 (mm) |
| Scanning | |
| drive axis = | 2Theta/Theta |
| scan range = | 2.507-39.987 |
| scan mode = | Continuous Scan |
| scan speed = | 0.0040 (deg/min) |
| sampling pitch = | 0.0200 (deg) |
| preset time = | 300.00 (sec) |

Data Process Condition:

| | |
|---|---|
| Smoothing | [AUTO] |
| smoothing points = | 19 |
| B.G. Subtraction | [AUTO] |
| sampling points = | 21 |
| repeat times = | 30 |
| Ka1-a2 Separate | [MANUAL] |
| Ka1 a2 ratio = | 50.0 (%) |
| Peak Search | [AUTO] |
| differential points = | 17 |
| FWHM threshold = | 0.050 (deg) |
| intensity threshold = | 30 (par mil) |
| FWHM ratio (n − 1)/n = | 2 |
| System Error Correction: | [NO] |
| Precise Peak Correction: | [NO] |

TABLE 13
Peak Positions of Ilaprazole(−), Form O XRPD Pattern

| Peak | Position | | | |
|---|---|---|---|---|
| No. | (°2θ) | d-spacing | Intensity | I/Iₒ |
| 1 | 7.6 | 11.6 | 66 | 4 |
| 2 | 7.9 | 11.1 | 324 | 19 |
| 3 | 10.0 | 8.8 | 669 | 39 |
| 4 | 11.5 | 7.7 | 116 | 7 |
| 5 | 12.2 | 7.3 | 587 | 34 |
| 6 | 14.2 | 6.2 | 119 | 7 |
| 7 | 15.1 | 5.9 | 208 | 12 |
| 8 | 15.9 | 5.6 | 259 | 15 |
| 9 | 16.3 | 5.4 | 458 | 27 |
| 10 | 18.4 | 4.8 | 1718 | 100 |
| 11 | 19.3 | 4.6 | 219 | 13 |
| 12 | 20.1 | 4.4 | 191 | 11 |
| 13 | 21.4 | 4.1 | 1249 | 73 |
| 14 | 21.8 | 4.1 | 1480 | 86 |
| 15 | 22.9 | 3.9 | 324 | 19 |
| 16 | 24.0 | 3.7 | 191 | 11 |
| 17 | 24.6 | 3.6 | 1277 | 74 |
| 18 | 25.0 | 3.6 | 164 | 10 |
| 19 | 26.6 | 3.4 | 209 | 12 |
| 20 | 26.8 | 3.3 | 186 | 11 |
| 21 | 28.0 | 3.2 | 70 | 4 |
| 22 | 28.5 | 3.1 | 225 | 13 |
| 23 | 28.9 | 3.1 | 731 | 43 |
| 24 | 29.3 | 3.0 | 76 | 4 |
| 25 | 29.8 | 3.0 | 266 | 15 |
| 26 | 30.2 | 3.0 | 142 | 8 |
| 27 | 30.4 | 2.9 | 142 | 8 |
| 28 | 31.0 | 2.9 | 175 | 10 |
| 29 | 35.1 | 2.6 | 61 | 4 |
| 30 | 35.8 | 2.5 | 116 | 7 |
| 31 | 38.7 | 2.3 | 61 | 4 |

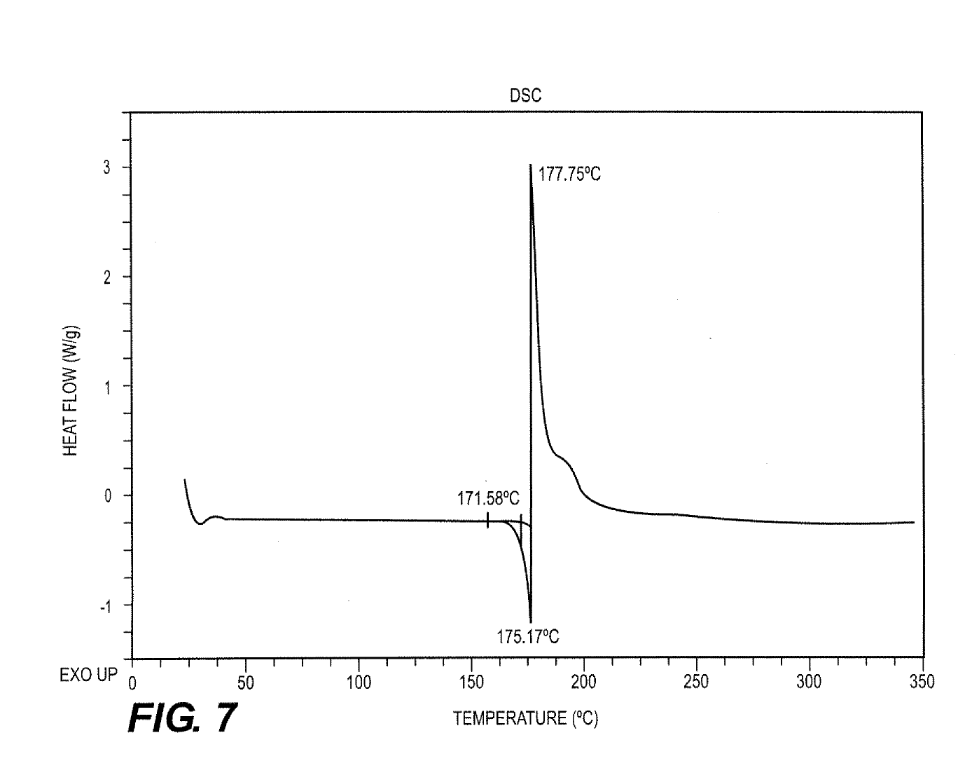

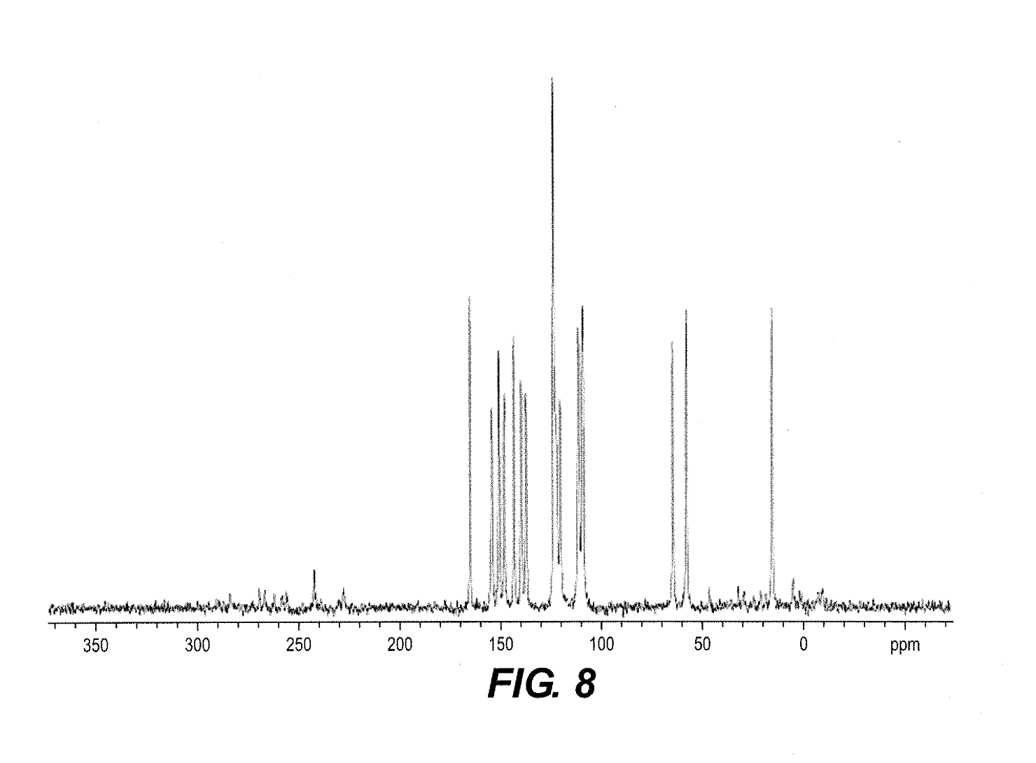

FIG. 7 is the DSC thermogram of Ilaprazole(−), Form O. The endotherm onset was 171° C. (max 175° C.). FIG. 8 is the solid state ¹³C CP/MAS NMR of Ilaprazole(−), Form O, externally referenced against glycine at 176.5 ppm. Table 12 lists the ¹³C NMR peaks for ilaprazole form O(−).

TABLE 14
Solid state ¹³C NMR peaks of Ilaprazole(−), Form O

| δ ppm | Height |
|---|---|
| 164.3 | 83.0 |
| 153.5 | 53.1 |
| 149.9 | 68.4 |
| 147.2 | 57.1 |
| 142.4 | 72.2 |
| 138.8 | 60.8 |
| 136.4 | 57.2 |
| 122.7 | 141.8 |
| 119.3 | 55.6 |
| 110.2 | 74.6 |
| 107.9 | 80.5 |
| 63.1 | 70.9 |
| 56.4 | 79.6 |
| 13.8 | 80.0 |

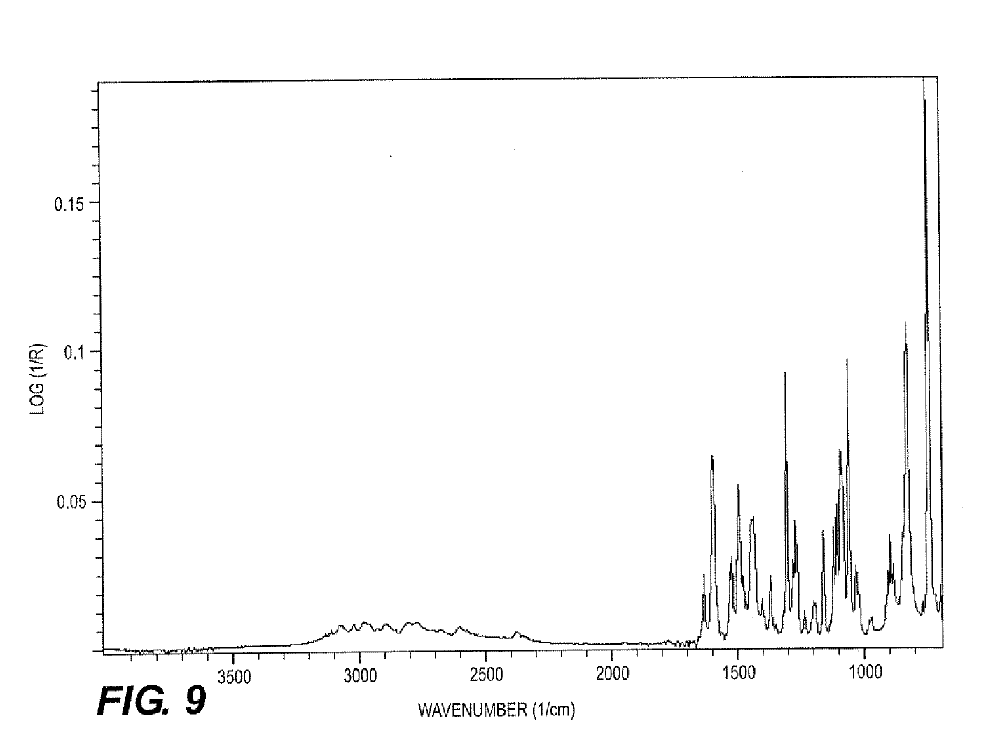

FIG. 9 is the IR spectrum of Ilaprazole(−), Form O. Table 15 lists the IR peaks.

TABLE 15
Peak Positions of Ilaprazole(−), Form O IR Spectrum

| | Intensity |
|---|---|
| Position | (Log |
| (cm⁻¹) | (1/R)) |
| 733 | 0.191 |
| 760 | 0.0155 |
| 818 | 0.11 |
| 837 | 0.0384 |
| 874 | 0.0278 |
| 885 | 0.0379 |
| 894 | 0.0261 |
| 959 | 0.0105 |
| 1011 | 0.0186 |
| 1021 | 0.0278 |
| 1049 | 0.0964 |
| 1071 | 0.0606 |
| 1079 | 0.0666 |
| 1097 | 0.0481 |
| 1109 | 0.0402 |
| 1122 | 0.0092 |
| 1149 | 0.0389 |
| 1186 | 0.0156 |
| 1224 | 0.0129 |
| 1259 | 0.0423 |
| 1269 | 0.0292 |
| 1294 | 0.0916 |
| 1308 | 0.0127 |
| 1337 | 0.0079 |
| 1358 | 0.0244 |
| 1391 | 0.0167 |
| 1424 | 0.044 |
| 1430 | 0.0428 |
| 1455 | 0.0165 |
| 1467 | 0.0248 |
| 1481 | 0.0546 |
| 1512 | 0.031 |
| 1518 | 0.0265 |
| 1583 | 0.0642 |
| 1622 | 0.0251 |
| 1764 | 0.003 |
| 2590 | 0.0078 |
| 2665 | 0.007 |
| 2758 | 0.0095 |
| 2795 | 0.0096 |
| 2881 | 0.009 |
| 2916 | 0.0076 |
| 2972 | 0.0097 |
| 3010 | 0.0091 |
| 3066 | 0.0089 |
| 3098 | 0.0071 |
| 3120 | 0.0059 |

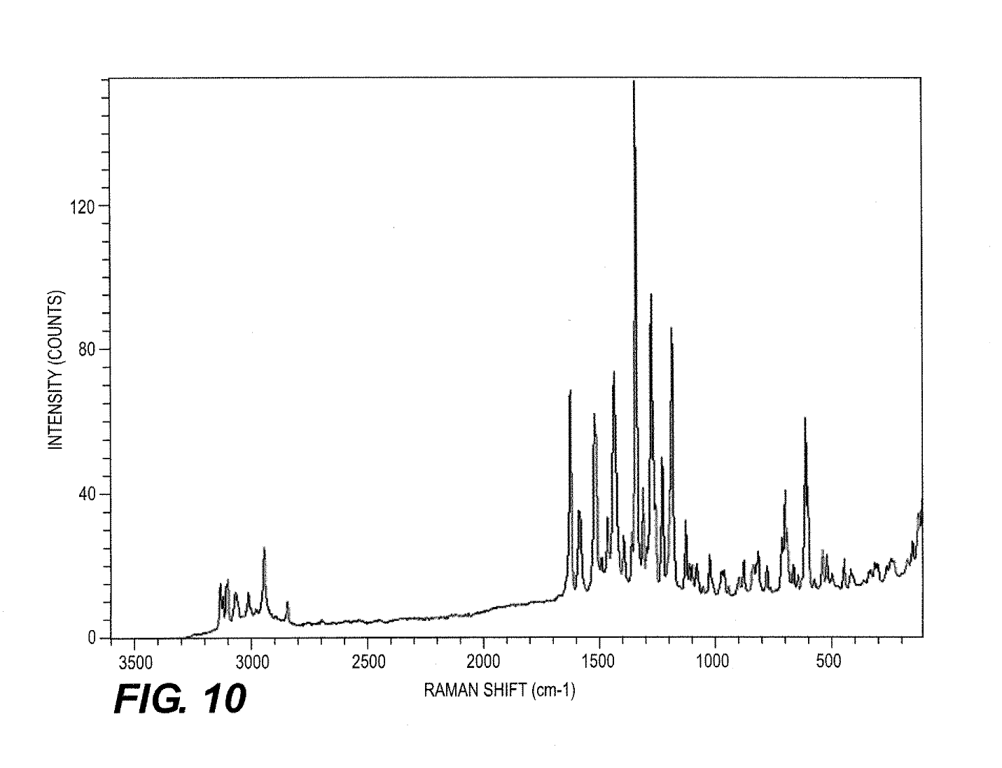

FIG. 10 is the Raman spectrum of Ilaprazole(−), Form O. Table 16 lists the Raman peaks.

TABLE 16
Peak Positions of Ilaprazole(−), Form O RAMAN Spectrum

| Position | |
|---|---|
| (cm⁻¹) | Intensity |
| 414 | 4.377 |
| 444 | 7.652 |
| 496 | 3.794 |
| 517 | 9.292 |
| 535 | 10.751 |
| 571 | 2.704 |
| 599 | 29.028 |
| 608 | 49.685 |
| 642 | 4.507 |
| 661 | 7.41 |
| 687 | 16.646 |
| 697 | 29.091 |
| 711 | 15.295 |
| 774 | 7.586 |
| 813 | 11.866 |
| 832 | 7.958 |
| 874 | 9.447 |
| 895 | 4.806 |
| 940 | 2.481 |
| 961 | 6.7 |
| 970 | 6.369 |
| 1021 | 11.421 |
| 1051 | 2.615 |
| 1077 | 9.051 |
| 1097 | 8.498 |
| 1109 | 9.091 |
| 1123 | 21.535 |
| 1182 | 74.4 |
| 1224 | 38.563 |
| 1255 | 25.553 |
| 1272 | 84.544 |
| 1292 | 13.517 |
| 1309 | 30.1 |
| 1337 | 145.988 |
| 1358 | 17.47 |
| 1391 | 16.798 |
| 1432 | 62.591 |
| 1463 | 22.061 |
| 1488 | 10.572 |
| 1512 | 47.318 |
| 1518 | 50.309 |
| 1585 | 24.167 |
| 1622 | 58.451 |
| 2843 | 6.969 |
| 2893 | 3.431 |
| 2943 | 22.986 |
| 2976 | 5.774 |
| 3011 | 10.995 |
| 3066 | 11.504 |
| 3099 | 15.424 |
| 3105 | 13.878 |
| 3120 | 10.595 |
| 3130 | 14.332 |

### Example 5

### Preparation of Amorphous Ilaprazole(−)

Approximately 24.5 mg ilaprazole(−), Form O was added to a solution containing 2 ml dichloromethane (DCM) and 30 μl triethylamine (TEA). The resulting clear solution was filtered through a 0.2 micron nylon filter into a glass vial containing approximately 10 ml cold hexanes. Immediate precipitation was observed and the sample was left in a dry ice/isopropanol (IPA) bath for approximately 5 minutes. The resulting white solid was collected by vacuum filtration as amorphous ilaprazole(−).

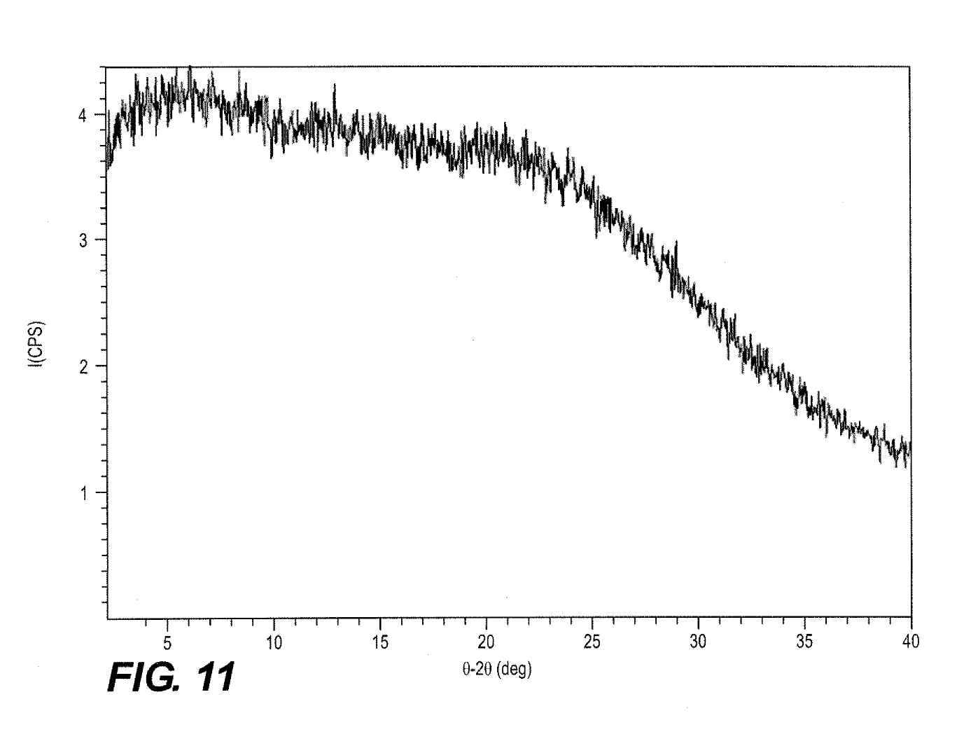

The XRPD pattern of amorphous ilaprazole(−) was obtained using an Inel XRG-3000 diffractometer. FIG. 11 is the XRPD pattern for amorphous ilaprazole(−). No peaks are seen indicating a non-crystalline, amorphous form of ilaprazole (−).

### Example 6

### Solubility Studies of Ilaprazole(−), Form A

Approximate solubilities of ilaprazole(−), Form A in two concentrations of solvents and base at ambient temperature were determined as part of the polymorph screen. The solubilities were calculated based on the total solvent used to give a solution; actual solubilities may be greater because of the volume of the solvent portions utilized or a slow rate of dissolution. Solubilities were rounded to the nearest mg/mL. Table 17 lists the approximate solubilities.

TABLE 17
Approximate Solubilities of Ilaprazole(−), Form A

| Solvent | Solubility (mg/mL) |
|---|---|
| 15:1 (w/w) EtOH:NH₄OH | 11 |
| (pH ~9) | |
| 10:1 (w/w) EtOH:NH₄OH | 20 |
| (pH ~9) | |

### Example 7

### Single Crystal Preparation

Crystals of Ilaprazole(−), Form A were prepared by acetone/hexanes vapor diffusion crystallization. The acetone had a small amount of triethylamine (TEA) added to stabilize the starting material. Crystals suitable for structure determination were observed after approximately one week.

### Data Collection

A colorless clear chunk of Ilaprazole(−), Form A, (empirical formula C₁₉H₁₈N₄O₂S) having approximate dimensions of 0.54×0.10×0.093 mm, was coated with Paratone N oil, suspended in a small fiber loop and placed in a cooled nitrogen gas stream in a random orientation. Preliminary examination and data collection were performed with Cu K_{α} radiation (λ==1.54178 Å) on a Bruker D8 APEX II CCD sealed tube diffractometer. Data collection, indexing and initial cell refinements were all carried out using APEX II software (APEX II, 2005, Bruker AXS, Inc., Analytical X-ray Systems, 5465 East Cheryl Parkway, Madison Wis. 53711-5373). Frame integration and final cell refinements were done using SAINT software (SAINT Version 6.45A, 2003, Bruker AXS, Inc., Analytical X-ray Systems, 5465 East Cheryl Parkway, Madison Wis. 53711-5373). The data were collected to a maximum 2θ value of 120.30°, at a temperature of 173±2 K.

Cell constants and an orientation matrix for data collection were obtained from least-squares refinement using the setting angles of 1270 reflections in the range 8.99°<θ<57.11°. The space group was determined by the program XPREP (Bruker, XPREP in SHELXTL v. 6.12, Bruker AXS Inc., Madison, Wis., USE, 2002). From the systematic presence of the following condition. 0k0 k=2n, and from subsequent least-squares refinement, the space group was determined to be P2₁ (no. 4).

### Data Reduction

The frames were collected using phi and omega scans. A total of 3480 reflections were collected, of which 2013 were unique. Lorentz and polarization corrections were applied to the data. The linear absorption coefficient is 18.2 cm⁻¹ for CuK_{α} radiation. A semi-empirical absorption correction using equivalents was applied. Intensities of equivalent reflections were averaged. The agreement factor for the averaging was 2.85% based on intensity.

### Structure Solution and Refinement

The structure was solved by direct methods using SHELXS-97 (Sheldrick, G. M. SHELX97, A Program for the Solution of Crystal Structure, University of Gottingen, Germany, 1997). The remaining atoms were located in succeeding difference Fourier syntheses using SHELX97 (Sheldrick, G. M. SHELX97, A Program for Crystal Structure Refinement, University of Gottingen, Germany, 1997). Hydrogen atoms were included in the refinement but restrained to ride on the atom to which they are bonded. The structure was refined in full-matrix least-squares by minimizing the function:

Σw(|Fₒ|²−|F_{c}|²)²

The weight w is defined as 1/[σ²(Fₒ²)+(0.0395 P)²+(0.0000 P)], where P=(Fₒ²+2F_{c}²)/3.

Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4). Of the 2013 reflections used in the refinements, only the reflections with Fₒ²>2σ(Fₒ²) were used in calculating R. A total of 1778 reflections were used in the calculation. The final cycle of refinement included variable parameters and converged (largest parameter shift was <0.01 times its estimated standard deviation) with unweighted and weighted agreement factors of:

R=Σ|Fₒ−F_{c}|/ΣFₒ=0.0364

R_{w}={square root over ((Σw(Fₒ²−F_{c}²)²/ρw(Fₒ²)²))}=0.0780

The standard deviation of an observation of unit weight was 1.054. The highest peak in the final difference Fourier had a height of 0.181 e/Å³. The minimum negative peak had a height of −0.229 e/Å³. The factor for the determination of the absolute structure (See Flack, H. D. Acta Cryst. 1983, A39, 876) refined to 0.05(2).

### Calculated X-Ray Powder Diffraction (XRPD) Pattern

A calculated XRPD pattern for (−) Ilaprazole Form A was generated for Cu radiation using PowderCell 2.3 (PowderCell for Windows Version 2.3 Kraus, W.; Nolze, G. Federal Institute for Materials Research and Testing, Berlin Germany, EU, 1999) and the atomic coordinates, space group, and unit cell parameters from the single crystal data.

### ORTEP and Packing Diagrams

The ORTEP diagram was prepared using ORTEP III (Johnson, C. K. ORTEPIII, Report ORNL-6895, Oak Ridge National Laboratory, TN, U.S.A. 1996; OPTEP-3 for Windows V1.05, Farrugia, L. J., J. Appl. Cryst. 1997, 30, 565). Atoms are represented by 50% probability anisotropic thermal ellipsoids. Packing diagrams were prepared using CAMERON (See, Watkin, D. J.; Prout, C. K.; Pearce, L. J. CAMERON, Chemical Crystallography Laboratory, University of Oxford, Oxford, 1996) modeling software.

### X-Ray Powder Diffraction (XRPD)

X-ray powder diffraction (XRPD) analyses were performed using an Inel XRG-3000 diffractometer equipped with a CPS (Curved Position Sensitive) detector with a 2θ range of 120°. Real time data were collected using Cu—Kα radiation starting at approximately 4° θ at a resolution of 0.03° 2θ. The tube voltage and amperage were set to 40 kV and 30 mA, respectively. The monochromator slit was set at 5 mm by 160 μm. The pattern is displayed from 2.5-40° 2θ. Samples were prepared for analysis by packing them into thin-walled glass capillaries. Each capillary was mounted onto a goniometer head that is motorized to permit spinning of the capillary during data acquisition. The samples were analyzed for 300 seconds. Instrument calibration was performed using a silicon reference standard. The experimental XRPD pattern was collected at SSCI, Inc. according to cGMP specifications.

### Results

The monoclinic cell parameters and calculated volume are: a=10.7759(4), b=7.3165(3), c=11.6182(4) Å, α=90.00, β=106.609(2), γ=90.00°, V=877.78(6) Å³. The molecular weight of Ilaprazole(−) molecule is 366.44 g/mol and with Z=2 the resulting in a calculated density of in the Form A crystal structure 1.386 g ⁻³. The space group was determined to be P2₁ (No. 4). This is a chiral space group. A summary of the crystal data and crystallographic data collection parameters are provided in Table 18.

TABLE 18
Crystal Data and Data Collection Parameters for (−) Ilaprazole Form A

| Identification code | 99487 | |
|---|---|---|
| Empirical formula | C₁₉H₁₈N₄O₂S | |
| Formula weight | 366.43 | |
| Temperature | 173(2) K | |
| Wavelength | 1.54178 Å | |
| Crystal system | Monoclinic | |
| Space group | P2(1) | |
| Unit cell dimensions | a = 10.7759(4) Å | α = 90° |
| | b = 7.3165(3) Å | β = 106.609(2)° |
| | c = 11.6182(4) Å | γ = 90° |

| | |
|---|---|
| Volume | 877.78(6) Å³ |
| Z | 2 |
| Density (calculated) | 1.386 Mg/m³ |
| Absorption coefficient | 1.820 mm⁻¹ |
| F(000) | 384 |
| Crystal size | 0.54 × 0.10 × 0.093 mm³ |
| Theta range for data collection | 8.99 to 60.15° |
| Index ranges | −11 ≦ h ≦ 11, −8 ≦ k ≦ |
| | 7, −11 ≦ 1 ≦ 13 |
| Reflections collected | 3480 |
| Independent reflections | 2013 [R(int) = 0.0285] |
| Completeness to theta = 60.15° | 90.7% |
| Absorption correction | Semi-empirical from equivalents |
| Refinement method | Full-matrix least-squares on F² |
| Data/restraints/parameters | 2013/1/296 |
| Goodness-of-fit on F² | 1.054 |
| Final R indices [I > 2sigma(I)] | R1 = 0.0364, wR2 = 0.0780 |
| R indices (all data) | R1 = 0.0464, wR2 = 0.0844 |
| Absolute structure parameter | 0.05(2)^{a} |
| Largest diff. peak and hole | 0.181 and −0.229 e · Å⁻³ |

^{a}Flack, H. D. Acta Cryst., 1983 A39, 876.

The quality of the structure obtained is high, as indicated by the R-value of 0.0364 (3.64%). Usually R-values in the range of 0.02 to 0.06 are quoted for the most reliably determined structures (See Glusker, Jenny Pickworth; Trueblood, Kenneth N. Crystal Structure Analysis: A Primer, 2^{nd} ed.; Oxford University press. New York, 1985, p. 87).

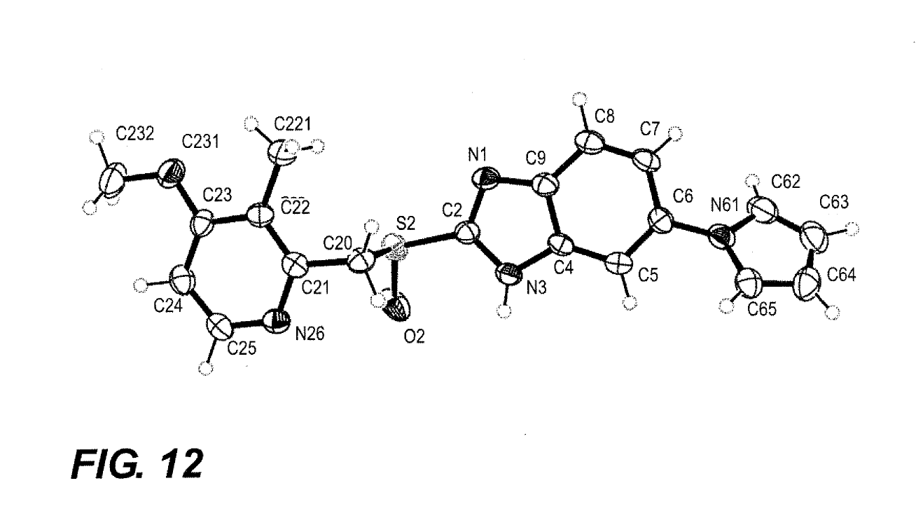

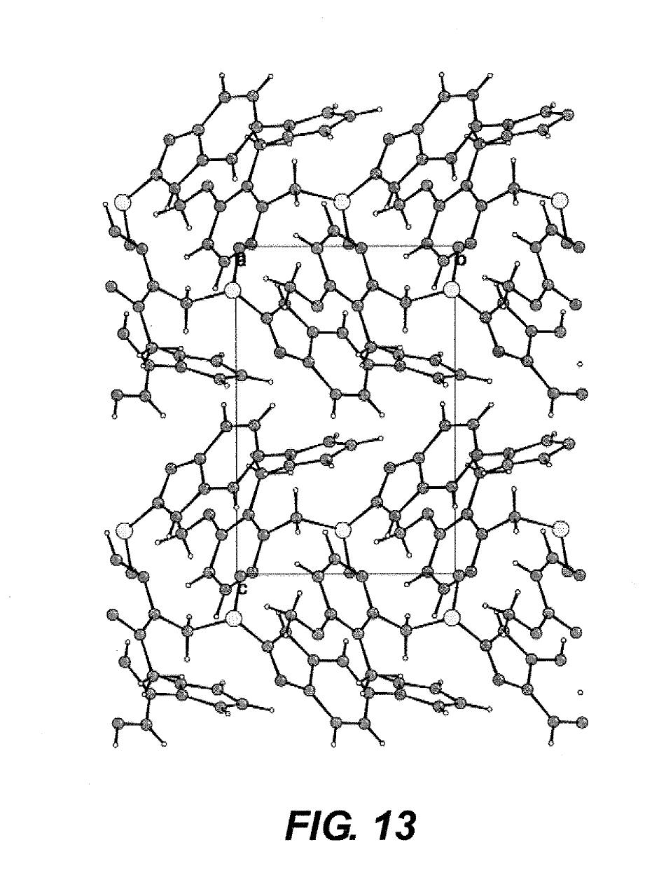

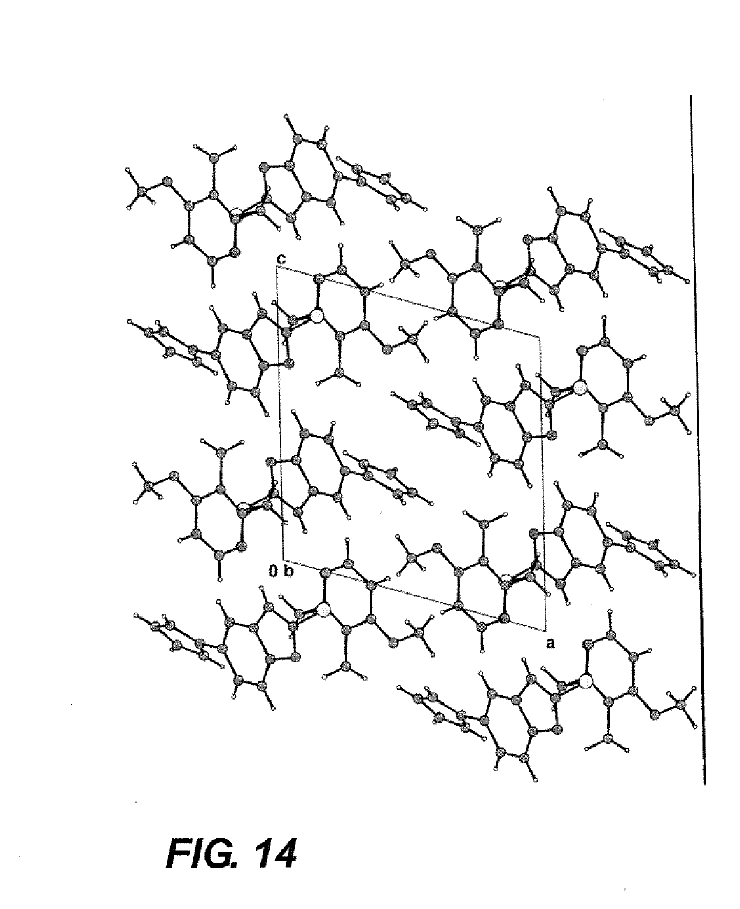

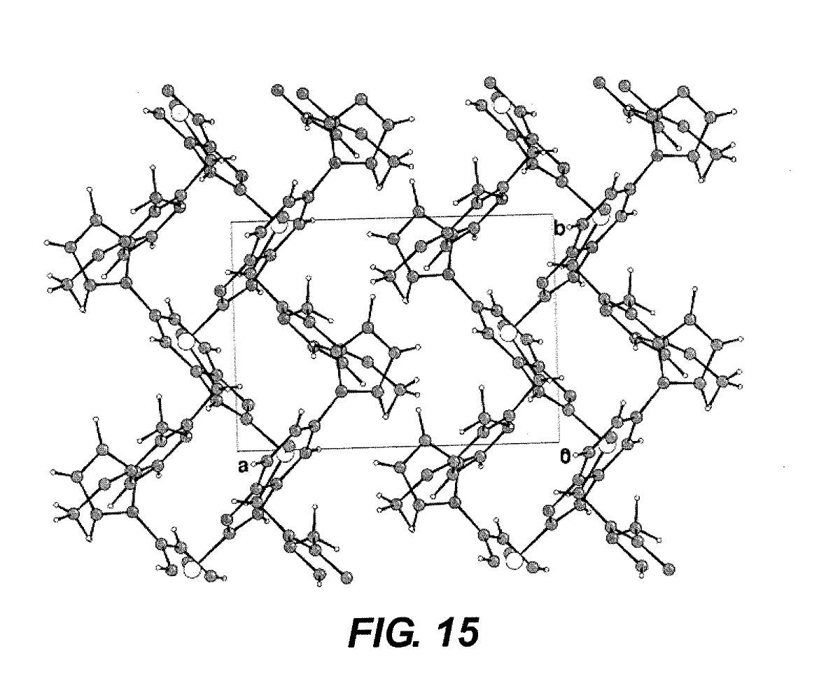

An ORTEP drawing of Ilaprazole(−), Form A is shown in FIG. 12. The asymmetric unit shown in FIG. 12 contains a single (−)Ilaprazole molecule. No disorder was observed in the sulfonyl oxygen atom. Packing diagrams viewed along the a, b, and c crystallographic axes are shown in FIGS. 13, 14, and 15, respectively. Hydrogen atoms are included in these figures. The packing arrangement consists of sheets of (−)Ilaprazole molecules running perpendicular to the crystallographic c axis (FIG. 15).

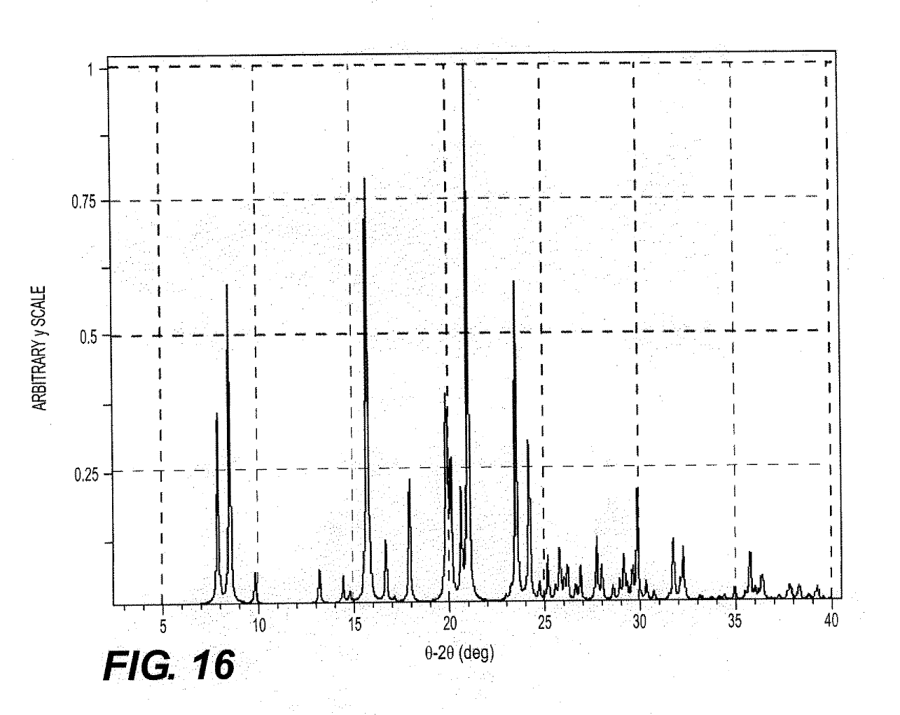

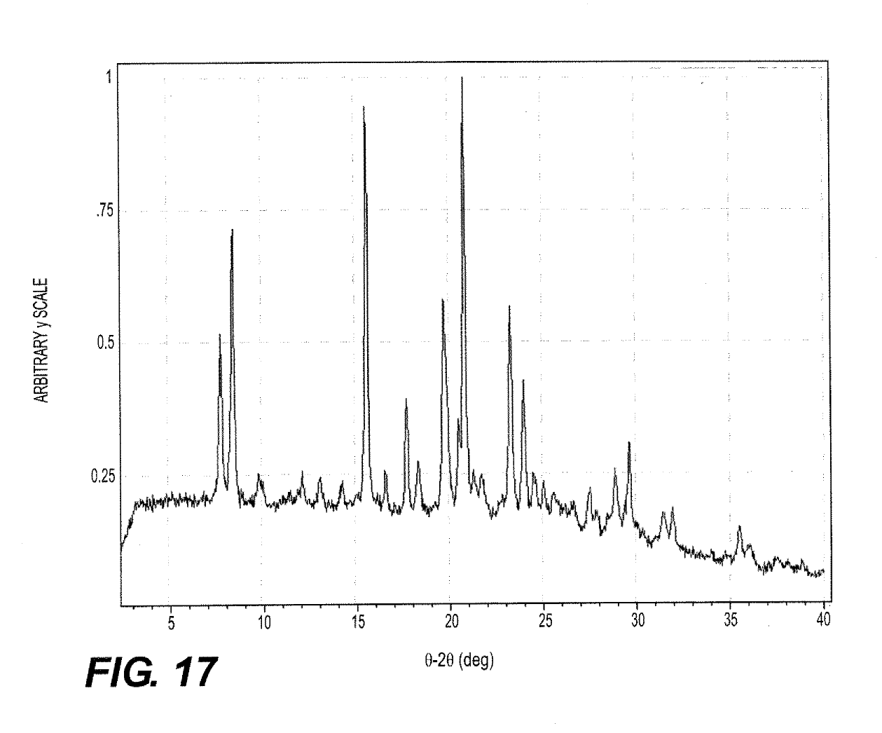

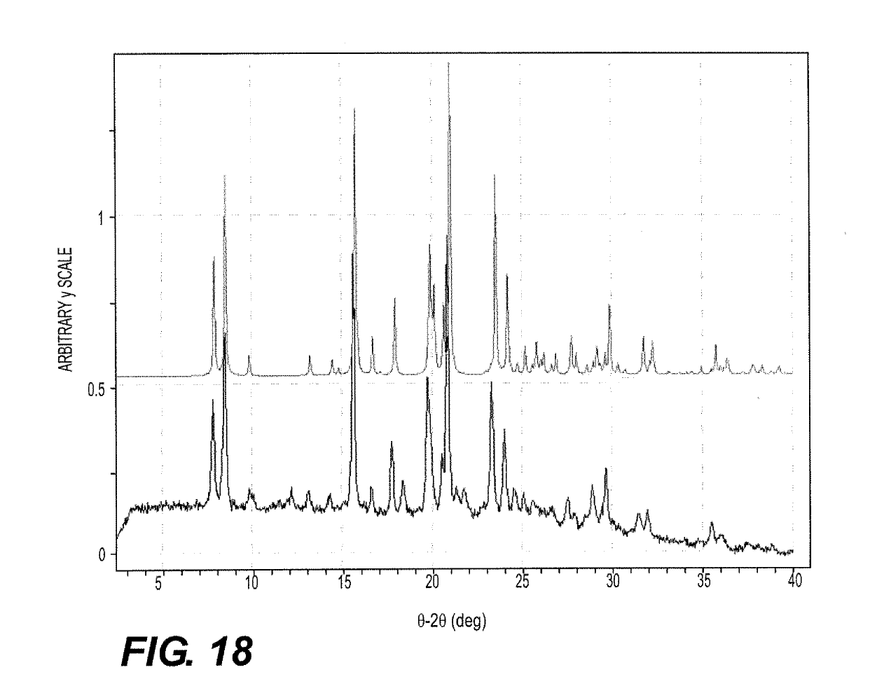

FIG. 16 shows a calculated XRPD pattern of Ilaprazole(−), Form A, generated from the single crystal data. The experimental XRPD pattern of Ilaprazole(−), Form A is shown in FIG. 17. FIG. 18 shows a comparison of the calculated and experimental XRPD patterns. All peaks in the experimental patterns are represented in the calculated XRPD pattern, indicating the bulk material is likely a single phase. The slight shifts in peak location are likely due to the fact that the experimental powder pattern was collected at ambient temperature, and the single crystal data was collected at 173 K. Low temperatures are used in single crystal analysis to improve the quality of the structure.

Because the material is a single enantiomer, the absolute configuration of the molecule can be determined by analysis of anomalous X-ray scattering by the crystal. The differences in intensities of the anomalous scattering are then compared with calculated scattering intensities for each enantiomer. These measured and calculated intensities can then be fit to a parameter, for instance, the Flack factor (See Flack, H. D.; Bernardinelli, G. Acta Cryst. 1999, A55, 908; Flack, H. D.; Bernardinelli, G. Reporting and evaluating absolute-structure and absolute-configuration determinations, J. Appl. Cryst. 2000, 33, 1143). The Flack factor, x(u) should be close to 0 if the configuration of the solved structure is correct, within statistical fluctuations, usually |x|<2u or x will be close to 1 if the inverse model is correct. The measured Flack factor for the structure of Ilaprazole(−), Form A shown in FIG. 13 is 0.05 with a standard uncertainty of 0.02 (Table 18). The standard uncertainty (u) is an indication of the inversion-distinguishing power, which is classified as strong/enantiopure-distinguishing. Therefore, the absolute configuration of the model in FIG. 13 is correct. This structure contains 1 chiral center located at S2, (see FIG. 13, ORTEP drawing), which has been assigned as S configuration. This is consistent with the proposed configuration in FIG. 12.

In sum, the single crystal structure of Ilaprazole(−), Form A was determined to confirm the molecular structure and to evaluate the occupancy of the sulfonyl oxygen. The space group was determined to be P2₁ (no. 4), which is a chiral space group. The structure of Ilaprazole Form A was successfully determined and no disorder was observed at the sulfonyl oxygen position. The chiral center at the S2 position was assigned as S configuration. The packing arrangement is essentially identical to the disordered mixed enantiomeric Form A crystal structure, indicating the material is a solid solution. All peaks in the calculated XRPD pattern are represented in the experimental pattern of Ilaprazole(−), Form A indicating the crystal is of the same form as the bulk material.

### Example 8

### Delayed Release Tablets

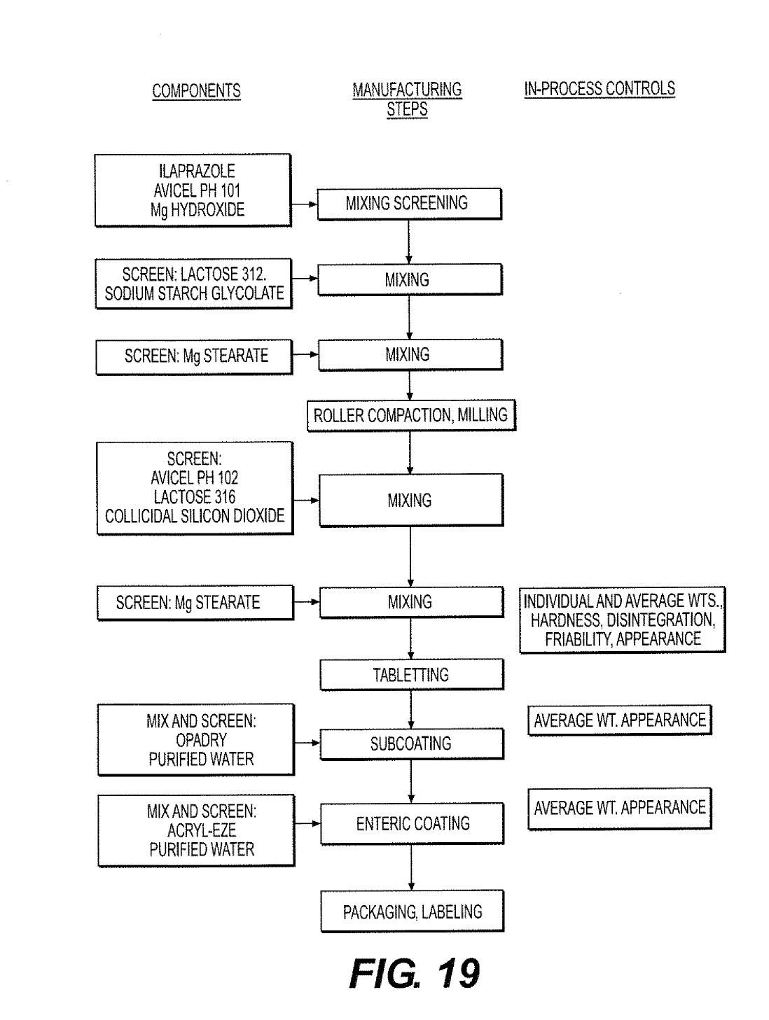

A representative batch size of ilaprazole delayed release tablets, 40 mg, may be prepared according to the representative batch formula show below in Table 19 and using the tableting process shown in FIG. 19.

TABLE 19
Target Composition of Delayed Release Tablets, 40 mg

| | | | | |
|---|---|---|---|---|
| | Quality | | | |
| Ingredient | Standard | Listed | Function | mg/tablet |

Core Tablet

| | | | | |
|---|---|---|---|---|
| Enantiopure Ilaprazole Form | Internal | — | Active | 40.00 |
| Magnesium Hydroxide | USP | IID | Stabilizer | 40.00 |
| Microcrystalline Cellulose (Avicel PH 101) | NF | IID | Diluent/Binder | 58.75 |
| Lactose Monohydrate (Foremost Lactose 312) | NF | IID | Diluent | 58.75 |
| Microcrystalline Cellulose (Avicel PH 102) | NF | IID | Diluent/Binder | 58.75 |
| Lactose Monohydrate (Foremost Fast-Flo 316) | NF | IID | Diluent | 58.75 |
| Sodium Starch Glycolate (Explotab) | NF | IID | Disintegrant | 12.14 |
| Colloidal Silicon Dioxide (Cab-O-Sil M5P) | NF | IID | Glidant | 0.8983 |
| Magnesium Stearate | NF | IID | Lubricant | 1.980 |

Subcoat

| | | | | |
|---|---|---|---|---|
| Opadry YS-1-19025-A Clear¹ | Internal | IID | Coating Material | 36.67 |
| Purified Water* | USP | N/A | Solvent | q.s. |

Enteric Coating

| | | | | |
|---|---|---|---|---|
| Acryl-EZE 93F19255 Clear² | Internal | — | Enteric Coating | 36.67 |
| Purified Water* | USP | N/A | Solvent | q.s. |
| Total | | | | 403.4 |

*Removed during processing.
IID - indicates use of the ingredient is supported by FDA Inactive Ingredient Database.
q.s. - sufficient quantity
N/A—not applicable, solvents are removed during processing.
¹Contains hypromellose, USP and polyethylene glycol 400, NF.
²Contains methacrylic acid copolymer type C, NF; polyethylene glycol 8000, NF; sodium bicarbonate, USP; colloidal anhydrous silica, NF; sodium lauryl sulfate, NF; and talc, USP.

The claimed invention is:

1. Crystalline enantiopure ilaprazole characterized by an x-ray powder diffraction pattern having characteristic peaks at 8.5° 2θ±0.2° 2θ and 13.1° 2θ±0.2° 2θ.

2. Crystalline enantiopure ilaprazole of claim 1, wherein the ilaprazole enantiomer is ilaprazole(+).

3. Crystalline enantiopure ilaprazole of claim 1, wherein the ilaprazole enantiomer is ilaprazole(−).

4. Crystalline enantiopure ilaprazole of claim 1, further characterized by an infrared spectrum having peaks at 712 cm⁻¹±1 cm⁻¹ and 776 cm⁻¹±1 cm⁻¹.

5. Crystalline enantiopure ilaprazole of claim 4, wherein the ilaprazole enantiomer is ilaprazole(+).

6. Crystalline enantiopure ilaprazole of claim 4, wherein the ilaprazole enantiomer is ilaprazole(−).

7. Crystalline enantiopure ilaprazole of claim 1, further characterized by a Raman spectrum having peaks at 448 cm⁻¹±1 cm⁻¹ and 625 cm⁻¹±1 cm⁻¹.

8. Crystalline enantiopure ilaprazole of claim 7, wherein the ilaprazole enantiomer is ilaprazole(+).

9. Crystalline enantiopure ilaprazole of claim 7, wherein the ilaprazole enantiomer is ilaprazole(−).

10. Crystalline enantiopure ilaprazole characterized by an x-ray powder diffraction pattern having characteristic peaks at 11.5 2θ±0.2° 2θ and 12.2° 2θ±0.2° 2θ.

11. Crystalline enantiopure ilaprazole of claim 10, wherein the ilaprazole enantiomer is ilaprazole(+).

12. Crystalline enantiopure ilaprazole of claim 10, wherein the ilaprazole enantiomer is ilaprazole(−).

13. Crystalline enantiopure ilaprazole of claim 10, further characterized by an infrared spectrum having peaks at 837 cm⁻¹±1 cm⁻¹ and 885 cm⁻¹±1 cm⁻¹.

14. Crystalline enantiopure ilaprazole of claim 13, wherein the ilaprazole enantiomer is ilaprazole(+).

15. Crystalline enantiopure ilaprazole of claim 13, wherein the ilaprazole enantiomer is ilaprazole(−).

16. Crystalline enantiopure ilaprazole of claim 10, further characterized by a Raman spectrum having peaks at 444 cm⁻¹±1 cm⁻¹ and 642 cm⁻¹±1 cm⁻¹.

17. Crystalline enantiopure ilaprazole of claim 16, wherein the ilaprazole enantiomer is ilaprazole(+).

18. Crystalline enantiopure ilaprazole of claim 16, wherein the ilaprazole enantiomer is ilaprazole(−).

19. Amorphous enantiopure ilaprazole(−).

20. A pharmaceutical composition for inhibiting gastric acid secretion, comprising a therapeutically effective amount of crystalline enantiopure ilaprazole of claim 1 and a pharmaceutically acceptable carrier.

21. A pharmaceutical composition for inhibiting gastric acid secretion, comprising a therapeutically effective amount of crystalline enantiopure ilaprazole of claim 10 and a pharmaceutically acceptable carrier.

22. A pharmaceutical composition for inhibiting gastric acid secretion, comprising a therapeutically effective amount of amorphous enantiopure ilaprazole(−) of claim 19 and a pharmaceutically acceptable carrier.

23. A method for treating a gastrointestinal inflammatory disorder in a mammal, comprising administering to a patient in need thereof a therapeutically effective amount of crystalline enantiopure ilaprazole of claim 1.

24. The method of claim 23, wherein the amount of ilaprazole administered ranges from about 0.001 mg/kg to about 50 mg/kg of subject body weight per day.

25. A method for treating a gastrointestinal inflammatory disorder in a mammal, comprising administering to a patient in need thereof a therapeutically effective amount of crystalline enantiopure ilaprazole of claim 10.

26. The method of claim 25, wherein the amount of ilaprazole administered ranges from about 0.001 mg/kg to about 50 mg/kg of subject body weight per day.

27. A method for treating a gastrointestinal inflammatory disorder in a mammal, comprising administering to a patient in need thereof a therapeutically effective amount of amorphous enantiopure ilaprazole of claim 19.

28. The method of claim 27, wherein the amount of ilaprazole administered ranges from about 0.001 mg/kg to about 50 mg/kg of subject body weight per day.
